(19) United States
(12) Patent Application Publication
(10) Pub. No.: US 2010/0168111 A1
(43) Pub. Date: Jul. 1, 2010

(54) POLYMORPHIC FORM OF 5 CHLORO N {[(5S) 2 OXO 3 [4 (3 OXOMORPHOLIN 4 YL)PHENYL]OXA-ZOLIDIN 5 YL]-METHYL}THIOPHENE 2 CARBOXAMIDE

(75) Inventors: Prabhudas Bodhuri, Brantford (CA); Gamini Weeratunga, Brantford (CA)

Correspondence Address: Apotex, Inc., 150 Signet Drive, Toronto, ON, M9L 1T9, CA

(73) Assignee: Apotex Pharmachem Inc., Brantford (CA)

(21) Appl. No.: 12/347,176

(22) Filed: Dec. 31, 2008

(51) Int. Cl. A61K 31/5377; C07D 413/14; A61P 7/02

(52) U.S. Cl. 514/236.8; 544/137

(57) ABSTRACT

A polymorphic form of rivaroxaban, 5-chloro-N-{[(5S)-2-oxo-3-[4-(3-oxomorpholin-4-yl)phenyl]oxa-zolidin-5-yl]-methyl}thiophene-2-carboxamide (termed form APO-A), processes for the preparation thereof, and compositions and formulations comprising form APO-A are provided. Also provided are compositions comprising a crystalline form of rivaroxaban and solvents selected from C3 to C6 ketones, C3 to C4 amides and mixtures thereof.

### TECHNICAL FIELD

The present invention relates to polymorphic forms of rivaroxaban and methods for the preparation thereof.

### BACKGROUND

Rivaroxaban (5-chloro-N-{([(5S)-2-oxo-3-[4-(3-oxomorpholin-4-yl)phenyl]oxa-zolidin-5-yl]-methyl}thiophene-2-carboxamide) is a low molecular weight, orally administrable anticoagulant drug. The pharmaceutical directly inhibits the active form of serine protease Factor Xa (FXa). Rivaroxaban can be used for the prevention and treatment of various thromboembolic diseases, in particular of deep vein thrombosis (DVT), pulmonary embolism (PE), myocardial infarct, angina pectoris, reocclusions and restenoses after angioplasty or aortocoronary bypass, cerebral stroke, transitory ischemic attacks, and peripheral arterial occlusive diseases.

Rivaroxaban is disclosed in WO 01/47919 and WO 2004/060887 and has the following structure:

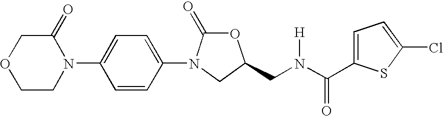

CA 2624310 relates to polymorphic forms and the amorphous form of (5-chloro-N-{[(5S)-2-oxo-3-[4-(3-oxomorpholin-4-yl)phenyl]oxa-zolidin-5-yl]-methyl}thiophene-2-carboxamide, methods for the production thereof, medicaments containing the same, and the use thereof for fighting diseases. Three modifications of rivaroxaban, namely modification I, II, and III are disclosed as well as an amorphous form, a hydrate, an NMP solvate and an inclusion compound with THF.

### SUMMARY

The present invention relates to a polymorphic form of the compound of formula (1), hereinafter referred to as form APO-A.

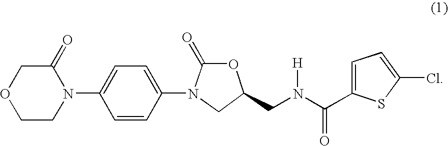

Form APO-A provides for reduced residual organic solvent in the crystalline form when compared to another polymorphic form of rivaroxaban.

Form APO-A may also exhibit increased solubility and thermal stability. Form APO-A may provide better oral bioavailability and/or a better dissolution profile for a particular formulation. Form APO-A may also provide free-flowing, easily filterable, and/or thermally stable characteristics that are suitable for use in particular formulations, for example and without limitation, liquid form formulations, solid form formulations, creams, gels, hydrogels, tablets, capsules and other known formulation forms.

In illustrative embodiments of the present invention, there is provided a polymorphic form of rivaroxaban characterized by an X-ray diffraction pattern having at least one peak in the X-ray diffraction pattern as set out in FIG. 1.

In illustrative embodiments of the present invention, there is provided a polymorphic form of rivaroxaban characterized by an X-ray diffraction pattern as set out in FIG. 1.

In illustrative embodiments of the present invention, there is provided a method of making form APO-A, a polymorphic form of rivaroxaban.

In illustrative embodiments of the present invention, there is provided a composition comprising form APO-A. In some embodiments, the composition is a pharmaceutical compositions comprising one or more pharmaceutically acceptable excipients.

In illustrative embodiments of the present invention, there is provided a composition comprising a crystalline form of rivaroxaban and an organic solvent selected from the group consisting of C3 to C6 ketones, C3 to C4 amides and mixtures thereof.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention,

FIG. 1 is a powder X-ray diffractogram (PXRD) (Cu—K.alpha) pattern of form APO-A.

FIG. 2 is a differential scanning calorimetry (DSC) thermogram of form APO-A.

### DETAILED DESCRIPTION

As used herein, the term “about” generally means within ±10%, often within ±5%, and often within ±1% of a given value or range and could be any increment thereof within ±10% (e.g. 0.1%, 0.5%, 1%, 2%, 3%, 4%, 5%, 6%, 7%, 8%, 9%, 10%, etc).

As used herein when referring to a spectrum and/or to data presented in a graph, the term “peak” refers a feature that one skilled in the art would recognize as not attributable to background noise.

As used herein, the term “polymorph” and the term “polymorphic form” refer to a crystallographically distinct form of a substance.

Different polymorphs of the same compound may have different physical, chemical, biological and/or spectroscopic properties. For example, and without limitation, different polymorphic forms may have different stability properties. A particular polymorphic form may be more sensitive to heat, relative humidity and/or light. Alternatively or additionally, a particular polymorphic form may provide more compressibility and/or density properties thereby providing more desirable characteristics for formulation and/or product manufacturing. Alternatively or additionally, a particular polymorphic form may have a different dissolution rate thereby providing more desirable bioavailability. In some cases, differences in stability result from changes in chemical reactivity, such as and without limitation, differential oxidation. Such properties may provide for more suitable product qualities such as a dosage form that is more resistant to discoloration when comprised of a particular polymorph. Mechanical characteristics of compounds may differ between polymorphs also. For example and without limitation, tablets having a higher ratio of a particular polymorph may be more resistant to crumbling on storage. Different physical properties of polymorphs may affect their processing. For example, and without limitation, a particular polymorph may be more likely to form solvates or may be more difficult to filter and/or wash.

Polymorphs of a molecule can be obtained by a number of methods known in the art. Such methods include, but are not limited to, recrystallization, melt recrystallization, melt cooling, solvent recrystallization (including using single or multiple solvents), precipitation, anti-solvent precipitation, evaporation, rapid evaporation, slurrying, slurry ripening, suspension equilibration, desolvation, dehydration, vapor diffusion, liquid-liquid diffusion, sublimation, grinding, milling, crystallization from the melt, heat induced transformations, desolvation of solvates, salting out, pH change, lyophilization, distillation, drying, rapid cooling, slow cooling, and combinations thereof.

Polymorphs can be detected, identified, classified and characterized using well-known techniques such as, but not limited to, differential scanning calorimetry (DSC), thermogravimetry (TGA), powder X-ray diffractometry (PXRD), single crystal X-ray diffractometry, vibrational spectroscopy, solution calorimetry, solid state nuclear magnetic resonance (NMR), infrared (IR) spectroscopy, Raman spectroscopy, hot stage optical microscopy, scanning electron microscopy (SEM), electron crystallography, quantitative analysis, solubility, and rate of dissolution.

In illustrative embodiments of the present invention, there is provided a polymorph of rivaroxaban hereinafter termed form APO-A of the compound of the formula (1):

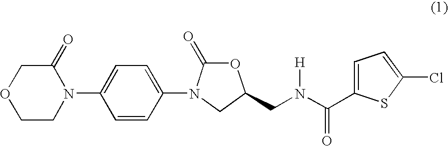

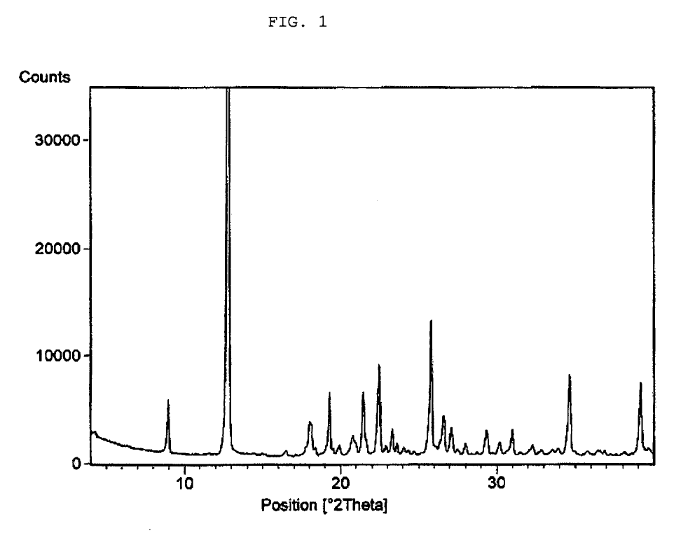

A powder X-ray diffractogram of form APO-A was produced as described in Example 3 and the diffractogram may be found in FIG. 1. Example 3 and FIG. 1 may be illustrative of the results that may be obtained when using diffraction of X-ray radiation to analyze form APO-A. As such, form APO-A may have a characteristic reflection (referenced in Tables 1 and 2 below as peak no. APO #) at any one or more of the values expressed in degrees 2 theta in Tables 1 and/or 2. Although values are given in the tables below, the polymorph is defined by the claimed peaks and a particular claim may be limited to one peak only, or several peaks. The form APO-A polymorph does not have to include all or even many of the peaks described in the tables that follow.

TABLE 1
1 Decimal Point ± up to 0.3
Peak No.

| | +0.3 | +0.2 | +0.1 | e.v. | −0.1 | −0.2 | −0.3 |
|---|---|---|---|---|---|---|---|
| APO 1 | 9.3 | 9.2 | 9.1 | 9.0 | 8.9 | 8.8 | 8.7 |
| APO 2 | 13.1 | 13.0 | 12.9 | 12.8 | 12.7 | 12.6 | 12.5 |
| APO 3 | 13.2 | 13.1 | 13.0 | 12.9 | 12.8 | 12.7 | 12.6 |
| APO 4 | 18.3 | 18.2 | 18.1 | 18.0 | 17.9 | 17.8 | 17.7 |
| APO 5 | 19.6 | 19.5 | 19.4 | 19.3 | 19.2 | 19.1 | 19.0 |
| APO 6 | 20.2 | 20.1 | 20.0 | 19.9 | 19.8 | 19.7 | 19.6 |
| APO 7 | 21.1 | 21.0 | 20.9 | 20.8 | 20.7 | 20.6 | 20.5 |
| APO 8 | 21.7 | 21.6 | 21.5 | 21.4 | 21.3 | 21.2 | 21.1 |
| APO 9 | 22.7 | 22.6 | 22.5 | 22.4 | 22.3 | 22.2 | 22.1 |
| APO 10 | 26.1 | 26.0 | 25.9 | 25.8 | 25.7 | 25.6 | 25.5 |
| APO 11 | 26.9 | 26.8 | 26.7 | 26.6 | 26.5 | 26.4 | 26.3 |
| APO 12 | 27.4 | 27.3 | 27.2 | 27.1 | 27.0 | 26.9 | 26.8 |
| APO 13 | 31.3 | 31.2 | 31.1 | 31.0 | 30.9 | 30.8 | 30.7 |
| APO 14 | 34.9 | 34.8 | 34.7 | 34.6 | 34.5 | 34.4 | 34.3 |
| APO 15 | 39.4 | 39.3 | 39.2 | 39.1 | 39.0 | 38.9 | 38.8 |

TABLE 2
2 Decimal Places ± up to 0.25

| | Peak No. | | | | | | | | | | |
|---|---|---|---|---|---|---|---|---|---|---|---|
| | +0.25 | +0.20 | +0.15 | +0.10 | +0.05 | e.v. | −0.05 | −0.10 | −0.15 | −0.20 | −0.25 |
| APO 1 | 9.22 | 9.17 | 9.12 | 9.07 | 9.02 | 8.97 | 8.92 | 8.87 | 8.82 | 8.77 | 8.72 |
| APO 2 | 13.06 | 13.01 | 12.96 | 12.91 | 12.86 | 12.81 | 12.76 | 12.71 | 12.66 | 12.61 | 12.56 |
| APO 3 | 13.10 | 13.05 | 13.00 | 12.95 | 12.90 | 12.85 | 12.80 | 12.75 | 12.70 | 12.65 | 12.60 |
| APO 4 | 18.24 | 18.19 | 18.14 | 18.09 | 18.04 | 17.99 | 17.94 | 17.89 | 17.84 | 17.79 | 17.74 |
| APO 5 | 19.51 | 19.46 | 19.41 | 19.36 | 19.31 | 19.26 | 19.21 | 19.16 | 19.11 | 19.06 | 19.01 |
| APO 6 | 20.14 | 20.09 | 20.04 | 19.99 | 19.94 | 19.89 | 19.84 | 19.79 | 19.74 | 19.69 | 19.64 |
| APO 7 | 21.01 | 20.96 | 20.91 | 20.86 | 20.81 | 20.76 | 20.71 | 20.66 | 20.61 | 20.56 | 20.51 |
| APO 8 | 21.69 | 21.64 | 21.59 | 21.54 | 21.49 | 21.44 | 21.39 | 21.34 | 21.29 | 21.24 | 21.19 |
| APO 9 | 22.69 | 22.64 | 22.59 | 22.54 | 22.49 | 22.44 | 22.39 | 22.34 | 22.29 | 22.24 | 22.19 |
| APO 10 | 26.03 | 25.98 | 25.93 | 25.88 | 25.83 | 25.78 | 25.73 | 25.68 | 25.63 | 25.58 | 25.53 |
| APO 11 | 26.81 | 26.76 | 26.71 | 26.66 | 26.61 | 26.56 | 26.51 | 26.46 | 26.41 | 26.36 | 26.31 |
| APO 12 | 27.34 | 27.29 | 27.24 | 27.19 | 27.14 | 27.09 | 27.04 | 26.99 | 26.94 | 26.89 | 26.84 |
| APO 13 | 31.21 | 31.16 | 31.11 | 31.06 | 31.01 | 30.96 | 30.91 | 30.86 | 30.81 | 30.76 | 30.71 |
| APO 14 | 34.85 | 34.80 | 34.75 | 34.70 | 34.65 | 34.60 | 34.55 | 34.50 | 34.45 | 34.40 | 34.35 |
| APO 15 | 39.37 | 39.32 | 39.27 | 39.22 | 39.17 | 39.12 | 39.07 | 39.02 | 38.97 | 38.92 | 38.87 |

Depending on the nature of the methodology applied and the scale selected to display results obtained from X-ray diffraction analysis, the peak intensities of peaks obtained my vary quite dramatically. For example, it is possible to obtain a relative peak intensity of 0.00% when analyzing one sample of a substance, but another sample of the same substance may show a much different relative intensity for a peak at the same position. This may be due, in part, to the relative orientation of the sample and its deviation from the preferred orientation of the sample, sample preparation and the methodology applied. Some illustrative and non-limiting possible observations regarding relative intensities of the peaks set out in Tables 1 and 2 above are set out below in Table 3.

TABLE 3
Relative Peak Intensities (Percent)

| | Peak No. | | | | | | |
|---|---|---|---|---|---|---|---|
| | +10% | +5 | +1 | e.v. | −1% | −5% | −10% |
| APO 1 | 16.30 | 11.30 | 7.30 | 6.30 | 5.30 | 1.30 | 0.00 |
| APO 2 | 100.00 | 100.00 | 100.00 | 100.00 | 99.00 | 95.00 | 90.00 |
| APO 3 | 60.00 | 55.00 | 51.00 | 50.00 | 49.00 | 45.00 | 40.00 |
| APO 4 | 13.91 | 8.91 | 4.91 | 3.91 | 2.91 | 0.00 | 0.00 |
| APO 5 | 17.42 | 12.42 | 8.42 | 7.42 | 6.42 | 2.42 | 0.00 |
| APO 6 | 11.27 | 6.27 | 2.27 | 1.27 | 0.27 | 0.00 | 0.00 |
| APO 7 | 12.23 | 7.23 | 3.23 | 2.23 | 1.23 | 0.00 | 0.00 |
| APO 8 | 16.68 | 11.68 | 7.68 | 6.68 | 5.68 | 1.68 | 0.00 |
| APO 9 | 20.64 | 15.64 | 11.64 | 10.64 | 9.64 | 5.64 | 0.64 |
| APO 10 | 24.62 | 19.62 | 15.62 | 14.62 | 13.62 | 9.62 | 4.62 |
| APO 11 | 14.16 | 9.16 | 5.16 | 4.16 | 3.16 | 0.00 | 0.00 |
| APO 12 | 13.25 | 8.25 | 4.25 | 3.25 | 2.25 | 0.00 | 0.00 |
| APO 13 | 12.89 | 7.89 | 3.89 | 2.89 | 1.89 | 0.00 | 0.00 |
| APO 14 | 18.45 | 13.45 | 9.45 | 8.45 | 7.45 | 3.45 | 0.00 |
| APO 15 | 18.72 | 13.72 | 9.72 | 8.72 | 7.72 | 3.72 | 0.00 |

In Tables 1, 2 and 3 above, the abbreviation “e.v.” stands for empirical value and represents a value obtained, in degrees 2 theta, empirically.

Information relating to the characteristics of a polymorphic or pseudopolymorphic form of a compound may be ascertained using X-ray crystallography. X-ray crystallography is also related to several other methods for determining atomic structures. Similar diffraction patterns can be produced by scattering electrons or neutrons, which are likewise interpreted as a Fourier transform.

X-ray crystallography may be used to determine the arrangement of atoms within a sample. This technique may be carried out using several different approaches. Common to all approaches is that a beam of X-rays is fired towards at least one crystal and/or crystallite (the at least one crystal and/or crystallite is a sample). Upon hitting the sample, the X-rays scatter in many different directions. The pattern of the scattering of the X-rays is recorded and from this recording the angles and intensities of the scattered X-rays may be determined. Once the angles and intensities are collected a crystallographer can determine physical properties of the sample, which in some cases is a three-dimensional picture of the electron density within the sample. Using an electron density map so produced, the positions of the atoms in the sample can then be determined, as well as their chemical bonds, their disorder and a variety of other information.

Various X-ray scattering methods can be applied to obtain physical information about the sample. Such methods include, without limitation, single crystal X-ray diffraction, fiber diffraction, powder diffraction (PXRD) and small-angle X-ray scattering (SAXS). In all these methods, the scattering is elastic and the scattered X-rays may have the same wavelength as the incoming X-ray. In some cases, these methods may provide information that is more or less detailed than another method yet can be related to each other by one or more characteristics, such as the d-spacing of a sample. Furthermore, data collected using the different types of X-ray methods can be inter-related using algorithms well know in the art, for example, obtaining a predicted powder pattern from single crystal data.

Powder X-ray diffraction, when combined with other computational techniques may be used to obtain exacting information on atomic arrangement within a particular polymorph or pseudopolymorph (structure solution from powder X-ray diffraction data). One method of analyzing such data is to evaluate the peaks obtained at particular angles in the experiment, which may be converted to d-spacings which are characteristic of the particular unit cell of the polymorph or pseudopolymorph, using Bragg's Law:

nλ=2d sin θ

where n is an integer, λ is the wavelength of light, θ relates to the angle that the beam impinges the sample and d is the d-spacing within the crystal.

The relative intensities of the peaks in a powder diffractogram are prone to larger variations than the peak positions. Each peak intensity results from diffractions from one or more d-spacing within the sample. For example, the particle size and shape properties of the sample may make it unlikely that the crystals or crystallites in the sample being analyzed are in an ideal orientation for use in a obtaining a PXRD. Some particular orientations of the crystal or crystallite in the holder may be more statistically likely and in conjunction, the d-spacings that can be viewed with such crystals or crystallites in these positions are more likely to produce more intense peaks. A person of skill in the art of crystallography understands the various different parameters and limitations regarding the comparability of different results obtained from different machines and/or using different X-ray scattering techniques and is able to interpret such differences.

There are a variety of machines available for performing X-ray crystallography in all of its variously described methods. For example, the following companies commonly manufacture many different machines for use in obtaining structural information from a variety of different samples: PANalytical, Bruker, Rigaku and Thermo as well as other companies. In many circumstances the exact result obtained may be affected by the specific machine used.

Thermal analysis methods are another set of methodologies that may be used to identify and characterize polymorphic forms. One thermal method is differential scanning calorimetry (DSC). DSC involves the measurement of the change of the difference in the heat flow to the sample and to a reference sample while the two samples are subjected to a controlled temperature program. DSC raw data shows heat flow plotted against temperature, and heat flow refers to the heat flux difference between the sample and the reference. Various DSC methodologies may be applied, for example and without limitation, temperature DSC, hyper-DSC, heat-flux DSC, modulated temperature DSC, Tzero DSC, DSC-TGA, DSC-TGA-IR and Ramen-DSC. Irrespective of the type of DSC instrument used, the type of information that may be obtained is uniform.

Other thermal methods may also be applied to obtain similar information to DSC results and they include, but are not limited to, differential thermal analysis (DTA), microthermal analysis, thermogravimetric analysis (TGA), and thermally stimulated current.

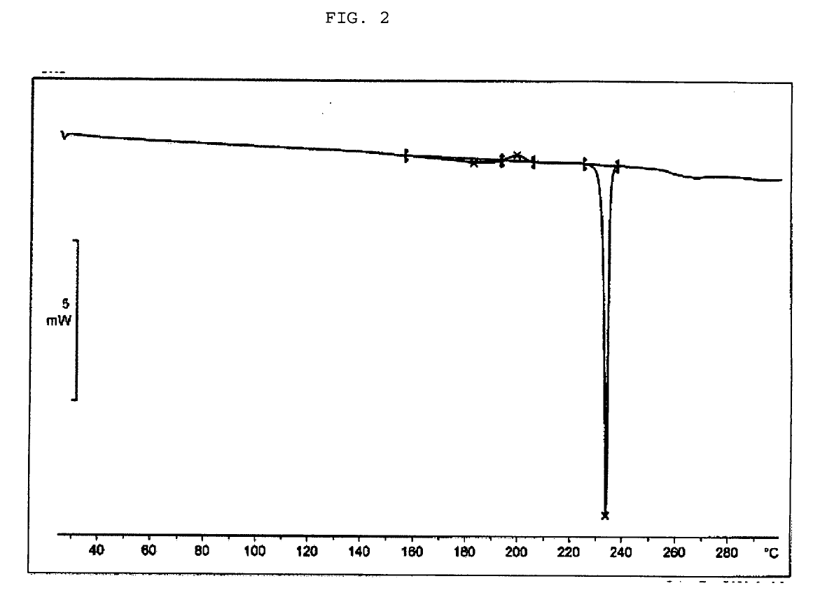

DSC of form APO-A was carried out as described in Example 4 and the thermogram may be found in FIG. 2. Example 4 and FIG. 2 may be illustrative of the results that may be obtained when using DSC to analyze form APO-A.

In illustrative embodiments of the present invention, there is provided a process for the preparation of form APO-A of the compound of the formula (1) comprising:
a. combining the compound of the formula (1) with a organic solvent or a mixture of solvents to form a mixture;
b. heating the mixture;
c. removing undissolved solid to form a solution;
d. promoting crystal growth thereby forming crystals; and
e. collecting crystals.

A compound of the formula (I) used in the process for the preparation of form APO-A described herein may be any form of rivaroxaban, including any polymorphic form of rivaroxaban, such as modification I.

A suitable organic solvent may be selected from the group consisting of C₃ to C₆ ketones such as 2-butanone, 3-pentanone, methyl isobutylketone, cyclohexanone; and C₃ to C₄ amides such as dimethyl formamide, dimethyl acetamide; and mixtures thereof.

The volume of the suitable organic solvent may be from about 8 to about 150 volumes. The volume of the suitable organic solvent may be from about 50 to about 130 volumes. The volume of the suitable organic solvent may be from about 80 to about 120 volumes.

The mixture may be heated to a temperature sufficient to obtain partial dissolution. The mixture may be heated to a temperature sufficient to obtain complete dissolution. The mixture may be heated to a temperature between about 20° C. to about 160° C. The mixture may be heated to a temperature between about 80° C., to about 120° C. The mixture may be heated to a temperature between about 100° C. to about 120° C.

Undissolved solid optionally may be removed by hot filtration of the mixture.

Crystal growth may be promoted by cooling the solution to a temperature between about 0° C. to about 50° C. Crystal growth may be promoted by cooling the solution to a temperature between about 0° C. to about 30° C. Crystal growth may be promoted by cooling the solution to a temperature between about 0° C. to about 15° C.

The crystals may be collected and/or purified by filtration. Drying, if desired, may also be carried out.

Form APO-A may be used in combination with other forms of rivaroxaban. Compositions comprising form APO-A and modification I are provided. Compositions comprising form APO-A and modification II are provided. Compositions comprising form APO-A and modification Ill are provided. Compositions comprising form APO-A and amorphous rivaroxaban are provided. Compositions comprising form APO-A, modification I and modification II are provided. Compositions comprising form APO-A, modification I and modification III are provided. Compositions comprising form APO-A, modification I and amorphous rivaroxaban are provided. Compositions comprising form APO-A, modification II and modification III are provided. Compositions comprising form APO-A, modification II and amorphous rivaroxaban are provided. Compositions comprising form APO-A, modification III and amorphous rivaroxaban are provided. Compositions comprising form APO-A, modification I, modification II and modification III are provided. Compositions comprising form APO-A, modification I, modification II and amorphous rivaroxaban are provided. Compositions comprising form APO-A, modification I, modification III and amorphous rivaroxaban are provided. Compositions comprising form APO-A, modification II, modification III and amorphous rivaroxaban are provided. Compositions comprising form APO-A, modification I, modification II, modification III and amorphous rivaroxaban are provided.

Compositions comprising form APO-A may comprise form APO-A in any quantity. Compositions may comprise from 1% or more form APO-A. Compositions may comprise 1% to 100% of form APO-A. Compositions may comprise 5% to 95% form APO-A. Compositions may comprise 10% to 95% form APO-A. Compositions may comprise 15% to 95% form APO-A. Compositions may comprise 20% to 95% form APO-A. Compositions may comprise 25% to 95% form APO-A. Compositions may comprise 30% to 95% form APO-A. Compositions may comprise 35% to 95% form APO-A. Compositions may comprise 40% to 95% form APO-A. Compositions may comprise 45% to 95% form APO-A. Compositions may comprise 50% to 95% form APO-A. Compositions may comprise 55% to 95% form APO-A. Compositions may comprise 60% to 95% form APO-A. Compositions may comprise 65% to 95% form APO-A. Compositions may comprise 70% to 95% form APO-A. Compositions may comprise 75% to 95% form APO-A. Compositions may comprise 80% to 95% form APO-A. Compositions may comprise 85% to 95% form APO-A. Compositions may comprise 90% to 95% form APO-A. Compositions may comprise 1% to 90% form APO-A. Compositions may comprise 1% to 85% form APO-A. Compositions may comprise 1% to 80% form APO-A. Compositions may comprise 1% to 75% form APO-A. Compositions may comprise 1% to 70% form APO-A. Compositions may comprise 1% to 65% form APO-A. Compositions may comprise 1% to 60% form APO-A. Compositions may comprise 1% to 55% form APO-A. Compositions may comprise 1% to 50% form APO-A. Compositions may comprise 1% to 45% form APO-A. Compositions may comprise 1% to 40% form APO-A. Compositions may comprise 1% to 35% form APO-A. Compositions may comprise 1% to 30% form APO-A. Compositions may comprise 1% to 25% form APO-A. Compositions may comprise 1% to 20% form APO-A. Compositions may comprise 1% to 15% form APO-A. Compositions may comprise 1% to 10% form APO-A. Compositions may comprise 1% to 5% form APO-A.

### EXAMPLES

### Example 1

### Preparation of 5-chloro-N-{[(5S)-2-oxo-3-[4-(3-oxomorpholin-4-yl)phenyl]oxa-zolidin-5-yl]-methyl}thiophene-2-carboxamide as form APO-A 300mg of

5-chloro-N-{[(5S)-2-oxo-3-[4-(3-oxomorpholin-4-yl)phenyl]oxa-zolidin-5-yl]-methyl}thiophene-2-carboxamide in the modification I was suspended in 40 mL of methyl-isobutylketone (MIBK), and 0.5 mL dimethylacetamide and heated to a temperature of 100° C. to 115° C. The resulting suspension was stirred at that temperature for 1 hour and filtered hot. The filtrate was cooled to room temperature and the solid was collected by filtration and dried at a temperature of 50° C.

### Example 2

### Preparation of 5-chloro-N-{[(5S)-2-oxo-3-[4-(3-oxomorpholin-4-yl)phenyl]oxa-zolidin-5-yl]-methyl}thiophene-2-carboxamide as form APO-A 300 mg of

5-chloro-N-{[(5S)-2-oxo-3-[4-(3-oxomorpholin-4-yl)phenyl]oxa-zolidin-5-yl]-methyl}thiophene-2-carboxamide in the modification I was suspended in 50 mL of methyl-isobutylketone (MIBK) and heated to a temperature of 100° C. to 115° C. The resulting suspension was stirred at that temperature for 1 hour and filtered hot. The filtrate was cooled to room temperature and the solid was collected by filtration and dried at a temperature of 50° C.

### Example 3

### X-ray Diffraction of Form APO-A

The X-ray powder diffraction patterns of the individual crystalline polymorphs prepared as described in Examples 1 and 2 were recorded with a PANalytical X'Pert Pro MPD diffractometer with fixed divergence slits and an X'Celerator RTMS detector. The diffractometer was configured in Bragg-Brentano geometry; data was collected over a 2 theta range of 4-40 using CuK.alpha radiation at a power of 40 mA and 45 kV. CuK.beta radiation was removed using a divergent beam nickel filter. A step size of 0.017 degrees and a step time of 30 seconds were used. Samples were rotated to reduce preferred orientation effects. Results are shown in FIG. 1.

### Example 4

### DSC of Form APO-A

DSC thermograms were collected on a Mettler-Toledo 821e instrument. Samples were weighed into a 40 uL aluminum pan and were crimped closed with an aluminum lid containing a 50 um pinhole. The samples were analyzed under a flow of nitrogen at a scan rate of 10° C./minute. Results are shown in FIG. 2.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes more than one such thing. Citation of references herein is not an admission that such references are prior art to the present invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

What is claimed is:

1. A polymorphic form of rivaroxaban characterized by an X-ray diffraction pattern comprising a peak at about 39.12 degrees two-theta.

2. A polymorphic form of rivaroxaban characterized by an X-ray diffraction pattern comprising a peak at 39.12 degrees two-theta plus or minus 0.2 degree two-theta.

3. A polymorphic form of rivaroxaban characterized by an X-ray diffraction pattern comprising a peak at 39.12 degrees two-theta.

4. A polymorphic form of rivaroxaban characterized by an X-ray diffraction pattern comprising a peak at about 34.60 degrees two-theta.

5. A polymorphic form of rivaroxaban characterized by an X-ray diffraction pattern comprising a peak at 34.60 degrees two-theta plus or minus 0.2 degree two-theta.

6. A polymorphic form of rivaroxaban characterized by an X-ray diffraction pattern comprising a peak at 34.60 degrees two-theta.

7. The polymorphic form of claim 1 wherein the X-ray diffraction pattern further comprises a peak at about 34.60 degrees two-theta.

8. The polymorphic form of claim 1 wherein the X-ray diffraction pattern further comprises a peak at 34.60 degrees two-theta plus or minus 0.2 degree two-theta.

9. The polymorphic form of claim 1 wherein the X-ray diffraction pattern further comprises a peak at 34.60 degrees two-theta.

10. The polymorphic form of claim 2 wherein the X-ray diffraction pattern further comprises a peak at about 34.60 degrees two-theta.

11. The polymorphic form of claim 2 wherein the X-ray diffraction pattern further comprises a peak at 34.60 degrees two-theta plus or minus 0.2 degree two-theta.

12. The polymorphic form of claim 2 wherein the X-ray diffraction pattern further comprises a peak at 34.60 degrees two-theta.

13. The polymorphic form of claim 3 wherein the X-ray diffraction pattern further comprises a peak at about 34.60 degrees two-theta.

14. The polymorphic form of claim 3 wherein the X-ray diffraction pattern further comprises a peak at 34.60 degrees two-theta plus or minus 0.2 degree two-theta.

15. The polymorphic form of claim 3 wherein the X-ray diffraction pattern further comprises a peak at 34.60 degrees two-theta.

16. The polymorphic form of claim 1 wherein the X-ray diffraction pattern further comprises peaks at about 25.78 degrees two-theta, about 22.44 degrees two-theta, about 19.27 degrees two-theta and about 8.97 degrees two-theta.

17. The polymorphic form of claim 1 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

18. The polymorphic form of claim 1 wherein the X-ray diffraction pattern further comprises peaks at 25.78 degrees two-theta, 22.44 degrees two-theta, 19.27 degrees two-theta and 8.97 degrees two-theta.

19. The polymorphic form of claim 2 wherein the X-ray diffraction pattern further comprises peaks at about 25.78 degrees two-theta, about 22.44 degrees two-theta, about 19.27 degrees two-theta and about 8.97 degrees two-theta.

20. The polymorphic form of claim 2 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

21. The polymorphic form of claim 2 wherein the X-ray diffraction pattern further comprises peaks at 25.78 degrees two-theta, 22.44 degrees two-theta, 19.27 degrees two-theta and 8.97 degrees two-theta.

22. The polymorphic form of claim 3 wherein the X-ray diffraction pattern further comprises peaks at about 25.78 degrees two-theta, about 22.44 degrees two-theta, about 19.27 degrees two-theta and about 8.97 degrees two-theta.

23. The polymorphic form of claim 3 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

24. The polymorphic form of claim 3 wherein the X-ray diffraction pattern further comprises peaks at 25.78 degrees two-theta, 22.44 degrees two-theta, 19.27 degrees two-theta and 8.97 degrees two-theta.

25. The polymorphic form of claim 4 wherein the X-ray diffraction pattern further comprises peaks at about 25.78 degrees two-theta, about 22.44 degrees two-theta, about 19.27 degrees two-theta and about 8.97 degrees two-theta.

26. The polymorphic form of claim 4 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

27. The polymorphic form of claim 4 wherein the X-ray diffraction pattern further comprises peaks at 25.78 degrees two-theta, 22.44 degrees two-theta, 19.27 degrees two-theta and 8.97 degrees two-theta.

28. The polymorphic form of claim 5 wherein the X-ray diffraction pattern further comprises peaks at about 25.78 degrees two-theta, about 22.44 degrees two-theta, about 19.27 degrees two-theta and about 8.97 degrees two-theta.

29. The polymorphic form of claim 5 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

30. The polymorphic form of claim 5 wherein the X-ray diffraction pattern further comprises peaks at 25.78 degrees two-theta, 22.44 degrees two-theta, 19.27 degrees two-theta and 8.97 degrees two-theta.

31. The polymorphic form of claim 6 wherein the X-ray diffraction pattern further comprises peaks at about 25.78 degrees two-theta, about 22.44 degrees two-theta, about 19.27 degrees two-theta and about 8.97 degrees two-theta.

32. The polymorphic form of claim 6 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

33. The polymorphic form of claim 6 wherein the X-ray diffraction pattern further comprises peaks at 25.78 degrees two-theta, 22.44 degrees two-theta, 19.27 degrees two-theta and 8.97 degrees two-theta.

34. The polymorphic form of claim 7 wherein the X-ray diffraction pattern further comprises peaks at about 25.78 degrees two-theta, about 22.44 degrees two-theta, about 19.27 degrees two-theta and about 8.97 degrees two-theta.

35. The polymorphic form of claim 7 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

36. The polymorphic form of claim 7 wherein the X-ray diffraction pattern further comprises peaks at 25.78 degrees two-theta, 22.44 degrees two-theta, 19.27 degrees two-theta and 8.97 degrees two-theta.

37. The polymorphic form of claim 8 wherein the X-ray diffraction pattern further comprises peaks at about 25.78 degrees two-theta, about 22.44 degrees two-theta, about 19.27 degrees two-theta and about 8.97 degrees two-theta.

38. The polymorphic form of claim 8 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

39. The polymorphic form of claim 8 wherein the X-ray diffraction pattern further comprises peaks at 25.78 degrees two-theta, 22.44 degrees two-theta, 19.27 degrees two-theta and 8.97 degrees two-theta.

40. The polymorphic form of claim 9 wherein the X-ray diffraction pattern further comprises peaks at about 25.78 degrees two-theta, about 22.44 degrees two-theta, about 19.27 degrees two-theta and about 8.97 degrees two-theta.

41. The polymorphic form of claim 9 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

42. The polymorphic form of claim 9 wherein the X-ray diffraction pattern further comprises peaks at 25.78 degrees two-theta, 22.44 degrees two-theta, 19.27 degrees two-theta and 8.97 degrees two-theta.

43. The polymorphic form of claim 10 wherein the X-ray diffraction pattern further comprises peaks at about 25.78 degrees two-theta, about 22.44 degrees two-theta, about 19.27 degrees two-theta and about 8.97 degrees two-theta.

44. The polymorphic form of claim 10 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

45. The polymorphic form of claim 10 wherein the X-ray diffraction pattern further comprises peaks at 25.78 degrees two-theta, 22.44 degrees two-theta, 19.27 degrees two-theta and 8.97 degrees two-theta.

46. The polymorphic form of claim 11 wherein the X-ray diffraction pattern further comprises peaks at about 25.78 degrees two-theta, about 22.44 degrees two-theta, about 19.27 degrees two-theta and about 8.97 degrees two-theta.

47. The polymorphic form of claim 11 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

48. The polymorphic form of claim 11 wherein the X-ray diffraction pattern further comprises peaks at 25.78 degrees two-theta, 22.44 degrees two-theta, 19.27 degrees two-theta and 8.97 degrees two-theta.

49. The polymorphic form of claim 12 wherein the X-ray diffraction pattern further comprises peaks at about 25.78 degrees two-theta, about 22.44 degrees two-theta, about 19.27 degrees two-theta and about 8.97 degrees two-theta.

50. The polymorphic form of claim 12 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

51. The polymorphic form of claim 12 wherein the X-ray diffraction pattern further comprises peaks at 25.78 degrees two-theta, 22.44 degrees two-theta, 19.27 degrees two-theta and 8.97 degrees two-theta.

52. The polymorphic form of claim 13 wherein the X-ray diffraction pattern further comprises peaks at about 25.78 degrees two-theta, about 22.44 degrees two-theta, about 19.27 degrees two-theta and about 8.97 degrees two-theta.

53. The polymorphic form of claim 13 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

54. The polymorphic form of claim 13 wherein the X-ray diffraction pattern further comprises peaks at 25.78 degrees two-theta, 22.44 degrees two-theta, 19.27 degrees two-theta and 8.97 degrees two-theta.

55. The polymorphic form of claim 14 wherein the X-ray diffraction pattern further comprises peaks at about 25.78 degrees two-theta, about 22.44 degrees two-theta, about 19.27 degrees two-theta and about 8.97 degrees two-theta.

56. The polymorphic form of claim 14 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

57. The polymorphic form of claim 14 wherein the X-ray diffraction pattern further comprises peaks at 25.78 degrees two-theta, 22.44 degrees two-theta, 19.27 degrees two-theta and 8.97 degrees two-theta.

58. The polymorphic form of claim 15 wherein the X-ray diffraction pattern further comprises peaks at about 25.78 degrees two-theta, about 22.44 degrees two-theta, about 19.27 degrees two-theta and about 8.97 degrees two-theta.

59. The polymorphic form of claim 15 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

60. The polymorphic form of claim 15 wherein the X-ray diffraction pattern further comprises peaks at 25.78 degrees two-theta, 22.44 degrees two-theta, 19.27 degrees two-theta and 8.97 degrees two-theta.

61. A polymorphic form of rivaroxaban characterized by an X-ray diffraction pattern comprising a peak at about 39.12 degrees two-theta with a relative intensity of at least about 8.72%.

62. A polymorphic form of rivaroxaban characterized by an X-ray diffraction pattern comprising a peak at 39.12 degrees two-theta plus or minus 0.2 degree two-theta with a relative intensity of at least about 8.72%.

63. A polymorphic form of rivaroxaban characterized by an X-ray diffraction pattern comprising a peak at 39.12 degrees two-theta with a relative intensity of at least about 8.72%.

64. A polymorphic form of rivaroxaban characterized by an X-ray diffraction pattern comprising a peak at about 34.60 degrees two-theta with a relative intensity of at least about 8.45%.

65. A polymorphic form of rivaroxaban characterized by an X-ray diffraction pattern comprising a peak at 34.60 degrees two-theta plus or minus 0.2 degree two-theta with a relative intensity of at least about 8.45%.

66. A polymorphic form of rivaroxaban characterized by an X-ray diffraction pattern comprising a peak at 34.60 degrees two-theta with a relative intensity of at least about 8.45%.

67. The polymorphic form of claim 61 wherein the X-ray diffraction pattern further comprises a peak at about 34.60 degrees two-theta with a relative intensity of at least about 8.45%.

68. The polymorphic form of claim 61 wherein the X-ray diffraction pattern further comprises a peak at 34.60 degrees two-theta plus or minus 0.2 degree two-theta with a relative intensity of at least about 8.45%.

69. The polymorphic form of claim 61 wherein the X-ray diffraction pattern further comprises a peak at 34.60 degrees two-theta with a relative intensity of at least about 8.45%.

70. The polymorphic form of claim 62 wherein the X-ray diffraction pattern further comprises a peak at about 34.60 degrees two-theta with a relative intensity of at least about 8.45%.

71. The polymorphic form of claim 62 wherein the X-ray diffraction pattern further comprises a peak at 34.60 degrees two-theta plus or minus 0.2 degree two-theta with a relative intensity of at least about 8.45%.

72. The polymorphic form of claim 62 wherein the X-ray diffraction pattern further comprises a peak at 34.60 degrees two-theta with a relative intensity of at least about 8.45%.

73. The polymorphic form of claim 63 wherein the X-ray diffraction pattern further comprises a peak at about 34.60 degrees two-theta with a relative intensity of at least about 8.45%.

74. The polymorphic form of claim 63 wherein the X-ray diffraction pattern further comprises a peak at 34.60 degrees two-theta plus or minus 0.2 degree two-theta with a relative intensity of at least about 8.45%.

75. The polymorphic form of claim 63 wherein the X-ray diffraction pattern further comprises a peak at 34.60 degrees two-theta with a relative intensity of at least about 8.45%.

76. The polymorphic form of claim 61 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

77. The polymorphic form of claim 61 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

78. The polymorphic form of claim 61 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

79. The polymorphic form of claim 62 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

80. The polymorphic form of claim 62 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

81. The polymorphic form of claim 62 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

82. The polymorphic form of claim 63 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

83. The polymorphic form of claim 63 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

84. The polymorphic form of claim 63 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

85. The polymorphic form of claim 64 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

86. The polymorphic form of claim 64 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

87. The polymorphic form of claim 64 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

88. The polymorphic form of claim 65 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

89. The polymorphic form of claim 65 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

90. The polymorphic form of claim 65 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

91. The polymorphic form of claim 66 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

92. The polymorphic form of claim 66 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

93. The polymorphic form of claim 66 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

94. The polymorphic form of claim 67 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

95. The polymorphic form of claim 67 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

96. The polymorphic form of claim 67 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

97. The polymorphic form of claim 68 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

98. The polymorphic form of claim 68 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

99. The polymorphic form of claim 68 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

100. The polymorphic form of claim 69 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

101. The polymorphic form of claim 69 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

102. The polymorphic form of claim 69 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

103. The polymorphic form of claim 70 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

104. The polymorphic form of claim 70 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

105. The polymorphic form of claim 70 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

106. The polymorphic form of claim 71 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

107. The polymorphic form of claim 71 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

108. The polymorphic form of claim 71 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

109. The polymorphic form of claim 72 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

110. The polymorphic form of claim 72 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

111. The polymorphic form of claim 72 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

112. The polymorphic form of claim 73 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

113. The polymorphic form of claim 73 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

114. The polymorphic form of claim 73 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

115. The polymorphic form of claim 74 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

116. The polymorphic form of claim 74 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

117. The polymorphic form of claim 74 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

118. The polymorphic form of claim 75 wherein the X-ray diffraction pattern further comprises peaks at about 25.78 degrees two-theta, about 22.44 degrees two-theta, about 19.27 degrees two-theta and about 8.97 degrees two-theta, each peak having a relative intensity of at least about 14.62%, at least about 10.64%, at least about 7.42% and at least about 6.30%, respectively.

119. The polymorphic form of claim 75 wherein the X-ray diffraction pattern further comprises peaks at 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta, each peak having a relative intensity of at least about 14.62%, at least about 10.64%, at least about 7.42% and at least about 6.30%, respectively.

120. The polymorphic form of claim 75 wherein the X-ray diffraction pattern further comprises peaks at 25.78 degrees two-theta, 22.44 degrees two-theta, 19.27 degrees two-theta and 8.97 degrees two-theta, each peak having a relative intensity of at least about 14.62%, at least about 10.64%, at least about 7.42% and at least about 6.30%, respectively.

121. The polymorph of claim 1 further characterized by a DSC thermogram exhibiting a peak endotherm at an onset temperature of about 166° C. and a peak maximum at about 183° C.

122. The polymorph of claim 2 further characterized by a DSC thermogram exhibiting a peak endotherm at an onset temperature of about 166° C. and a peak maximum at about 183° C.

123. The polymorph of claim 3 further characterized by a DSC thermogram exhibiting a peak endotherm at an onset temperature of about 166° C. and a peak maximum at about 183° C.

124. The polymorph of claim 4 further characterized by a DSC thermogram exhibiting a peak endotherm at an onset temperature of about 166° C. and a peak maximum at about 183° C.

125. The polymorph of claim 5 further characterized by a DSC thermogram exhibiting a peak endotherm at an onset temperature of about 166° C. and a peak maximum at about 183° C.

126. The polymorph of claim 6 further characterized by a DSC thermogram exhibiting a peak endotherm at an onset temperature of about 166° C. and a peak maximum at about 183° C.

127. The polymorph of claim 7 further characterized by a DSC thermogram exhibiting a peak endotherm at an onset temperature of about 166° C. and a peak maximum at about 183° C.

128. The polymorph of claim 8 further characterized by a DSC thermogram exhibiting a peak endotherm at an onset temperature of about 166° C. and a peak maximum at about 183° C.

129. The polymorph of claim 9 further characterized by a DSC thermogram exhibiting a peak endotherm at an onset temperature of about 166° C. and a peak maximum at about 183° C.

130. The polymorph of claim 10 further characterized by a DSC thermogram exhibiting a peak endotherm at an onset temperature of about 166° C. and a peak maximum at about 183° C.

131. The polymorph of claim 11 further characterized by a DSC thermogram exhibiting a peak endotherm at an onset temperature of about 166° C. and a peak maximum at about 183° C.

132. The polymorph of claim 12 further characterized by a DSC thermogram exhibiting a peak endotherm at an onset temperature of about 166° C. and a peak maximum at about 183° C.

133. The polymorph of claim 13 further characterized by a DSC thermogram exhibiting a peak endotherm at an onset temperature of about 166° C. and a peak maximum at about 183° C.

134. The polymorph of claim 14 further characterized by a DSC thermogram exhibiting a peak endotherm at an onset temperature of about 166° C. and a peak maximum at about 183° C.

135. The polymorph of claim 15 further characterized by a DSC thermogram exhibiting a peak endotherm at an onset temperature of about 166° C. and a peak maximum at about 183° C.

136. The polymorph of claim 16 further characterized by a DSC thermogram exhibiting a peak endotherm at an onset temperature of about 166° C. and a peak maximum at about 183° C.

137. The polymorph of claim 17 further characterized by a DSC thermogram exhibiting a peak endotherm at an onset temperature of about 166° C. and a peak maximum at about 183° C.

138. The polymorph of claim 18 further characterized by a DSC thermogram exhibiting a peak endotherm at an onset temperature of about 166° C. and a peak maximum at about 183° C.

139. The polymorph of claim 19 further characterized by a DSC thermogram exhibiting a peak endotherm at an onset temperature of about 166° C. and a peak maximum at about 183° C.

140. The polymorph of claim 20 further characterized by a DSC thermogram exhibiting a peak endotherm at an onset temperature of about 166° C. and a peak maximum at about 183° C.

141. The polymorph of claim 21 further characterized by a DSC thermogram exhibiting a peak endotherm at an onset temperature of about 166° C. and a peak maximum at about 183° C.

142. The polymorph of claim 22 further characterized by a DSC thermogram exhibiting a peak endotherm at an onset temperature of about 166° C. and a peak maximum at about 183° C.

143. The polymorph of claim 23 further characterized by a DSC thermogram exhibiting a peak endotherm at an onset temperature of about 166° C. and a peak maximum at about 183° C.

144. The polymorph of claim 24 further characterized by a DSC thermogram exhibiting a peak endotherm at an onset temperature of about 166° C. and a peak maximum at about 183° C.

145. The polymorph of claim 25 further characterized by a DSC thermogram exhibiting a peak endotherm at an onset temperature of about 166° C. and a peak maximum at about 183° C.

146. The polymorph of claim 26 further characterized by a DSC thermogram exhibiting a peak endotherm at an onset temperature of about 166° C. and a peak maximum at about 183° C.

147. The polymorph of claim 27 further characterized by a DSC thermogram exhibiting a peak endotherm at an onset temperature of about 166° C. and a peak maximum at about 183° C.

148. The polymorph of claim 28 further characterized by a DSC thermogram exhibiting a peak endotherm at an onset temperature of about 166° C. and a peak maximum at about 183° C.

149. The polymorph of claim 29 further characterized by a DSC thermogram exhibiting a peak endotherm at an onset temperature of about 166° C. and a peak maximum at about 183° C.

150. The polymorph of claim 30 further characterized by a DSC thermogram exhibiting a peak endotherm at an onset temperature of about 166° C. and a peak maximum at about 183° C.

151. The polymorph of claim 31 further characterized by a DSC thermogram exhibiting a peak endotherm at an onset temperature of about 166° C. and a peak maximum at about 183° C.

152. The polymorph of claim 32 further characterized by a DSC thermogram exhibiting a peak endotherm at an onset temperature of about 166° C. and a peak maximum at about 183° C.

153. The polymorph of claim 33 further characterized by a DSC thermogram exhibiting a peak endotherm at an onset temperature of about 166° C. and a peak maximum at about 183° C.

154. The polymorph of claim 34 further characterized by a DSC thermogram exhibiting a peak endotherm at an onset temperature of about 166° C. and a peak maximum at about 183° C.

155. The polymorph of claim 35 further characterized by a DSC thermogram exhibiting a peak endotherm at an onset temperature of about 166° C. and a peak maximum at about 183° C.

156. The polymorph of claim 36 further characterized by a DSC thermogram exhibiting a peak endotherm at an onset temperature of about 166° C. and a peak maximum at about 183° C.

157. The polymorph of claim 37 further characterized by a DSC thermogram exhibiting a peak endotherm at an onset temperature of about 166° C. and a peak maximum at about 183° C.

158. The polymorph of claim 38 further characterized by a DSC thermogram exhibiting a peak endotherm at an onset temperature of about 166° C. and a peak maximum at about 183° C.

159. The polymorph of claim 39 further characterized by a DSC thermogram exhibiting a peak endotherm at an onset temperature of about 166° C. and a peak maximum at about 183° C.

160. The polymorph of claim 40 further characterized by a DSC thermogram exhibiting a peak endotherm at an onset temperature of about 166° C. and a peak maximum at about 183° C.

161. The polymorph of claim 41 further characterized by a DSC thermogram exhibiting a peak endotherm at an onset temperature of about 166° C. and a peak maximum at about 183° C.

162. The polymorph of claim 42 further characterized by a DSC thermogram exhibiting a peak endotherm at an onset temperature of about 166° C. and a peak maximum at about 183° C.

163. The polymorph of claim 43 further characterized by a DSC thermogram exhibiting a peak endotherm at an onset temperature of about 166° C. and a peak maximum at about 183° C.

164. The polymorph of claim 44 further characterized by a DSC thermogram exhibiting a peak endotherm at an onset temperature of about 166° C. and a peak maximum at about 183° C.

165. The polymorph of claim 45 further characterized by a DSC thermogram exhibiting a peak endotherm at an onset temperature of about 166° C. and a peak maximum at about 183° C.

166. The polymorph of claim 46 further characterized by a DSC thermogram exhibiting a peak endotherm at an onset temperature of about 166° C. and a peak maximum at about 183° C.

167. The polymorph of claim 47 further characterized by a DSC thermogram exhibiting a peak endotherm at an onset temperature of about 166° C. and a peak maximum at about 183° C.

168. The polymorph of claim 48 further characterized by a DSC thermogram exhibiting a peak endotherm at an onset temperature of about 166° C. and a peak maximum at about 183° C.

169. The polymorph of claim 49 further characterized by a DSC thermogram exhibiting a peak endotherm at an onset temperature of about 166° C. and a peak maximum at about 183° C.

170. The polymorph of claim 50 further characterized by a DSC thermogram exhibiting a peak endotherm at an onset temperature of about 166° C. and a peak maximum at about 183° C.

171. The polymorph of claim 51 further characterized by a DSC thermogram exhibiting a peak endotherm at an onset temperature of about 166° C. and a peak maximum at about 183° C.

172. The polymorph of claim 52 further characterized by a DSC thermogram exhibiting a peak endotherm at an onset temperature of about 166° C. and a peak maximum at about 183° C.

173. The polymorph of claim 53 further characterized by a DSC thermogram exhibiting a peak endotherm at an onset temperature of about 166° C. and a peak maximum at about 183° C.

174. The polymorph of claim 54 further characterized by a DSC thermogram exhibiting a peak endotherm at an onset temperature of about 166° C. and a peak maximum at about 183° C.

175. The polymorph of claim 55 further characterized by a DSC thermogram exhibiting a peak endotherm at an onset temperature of about 166° C. and a peak maximum at about 183° C.

176. The polymorph of claim 56 further characterized by a DSC thermogram exhibiting a peak endotherm at an onset temperature of about 166° C. and a peak maximum at about 183° C.

177. The polymorph of claim 57 further characterized by a DSC thermogram exhibiting a peak endotherm at an onset temperature of about 166° C. and a peak maximum at about 183° C.

178. The polymorph of claim 58 further characterized by a DSC thermogram exhibiting a peak endotherm at an onset temperature of about 166° C. and a peak maximum at about 183° C.

179. The polymorph of claim 59 further characterized by a DSC thermogram exhibiting a peak endotherm at an onset temperature of about 166° C. and a peak maximum at about 183° C.

180. The polymorph of claim 60 further characterized by a DSC thermogram exhibiting a peak endotherm at an onset temperature of about 166° C. and a peak maximum at about 183° C.

181. The polymorph of claim 61 further characterized by a DSC thermogram exhibiting a peak endotherm at an onset temperature of about 166° C. and a peak maximum at about 183° C.

182. The polymorph of claim 62 further characterized by a DSC thermogram exhibiting a peak endotherm at an onset temperature of about 166° C. and a peak maximum at about 183° C.

183. The polymorph of claim 63 further characterized by a DSC thermogram exhibiting a peak endotherm at an onset temperature of about 166° C. and a peak maximum at about 183° C.

184. The polymorph of claim 64 further characterized by a DSC thermogram exhibiting a peak endotherm at an onset temperature of about 166° C. and a peak maximum at about 183° C.

185. The polymorph of claim 65 further characterized by a DSC thermogram exhibiting a peak endotherm at an onset temperature of about 166° C. and a peak maximum at about 183° C.

186. The polymorph of claim 66 further characterized by a DSC thermogram exhibiting a peak endotherm at an onset temperature of about 166° C. and a peak maximum at about 183° C.

187. The polymorph of claim 67 further characterized by a DSC thermogram exhibiting a peak endotherm at an onset temperature of about 166° C. and a peak maximum at about 183° C.

188. The polymorph of claim 68 further characterized by a DSC thermogram exhibiting a peak endotherm at an onset temperature of about 166° C. and a peak maximum at about 183° C.

189. The polymorph of claim 69 further characterized by a DSC thermogram exhibiting a peak endotherm at an onset temperature of about 166° C. and a peak maximum at about 183° C.

190. The polymorph of claim 70 further characterized by a DSC thermogram exhibiting a peak endotherm at an onset temperature of about 166° C. and a peak maximum at about 183° C.

191. The polymorph of claim 71 further characterized by a DSC thermogram exhibiting a peak endotherm at an onset temperature of about 166° C. and a peak maximum at about 183° C.

192. The polymorph of claim 72 further characterized by a DSC thermogram exhibiting a peak endotherm at an onset temperature of about 166° C. and a peak maximum at about 183° C.

193. The polymorph of claim 73 further characterized by a DSC thermogram exhibiting a peak endotherm at an onset temperature of about 166° C. and a peak maximum at about 183° C.

194. The polymorph of claim 74 further characterized by a DSC thermogram exhibiting a peak endotherm at an onset temperature of about 166° C. and a peak maximum at about 183° C.

195. The polymorph of claim 75 further characterized by a DSC thermogram exhibiting a peak endotherm at an onset temperature of about 166° C. and a peak maximum at about 183° C.

196. The polymorph of claim 76 further characterized by a DSC thermogram exhibiting a peak endotherm at an onset temperature of about 166° C. and a peak maximum at about 183° C.

197. The polymorph of claim 77 further characterized by a DSC thermogram exhibiting a peak endotherm at an onset temperature of about 166° C. and a peak maximum at about 183° C.

198. The polymorph of claim 78 further characterized by a DSC thermogram exhibiting a peak endotherm at an onset temperature of about 166° C. and a peak maximum at about 183° C.

199. The polymorph of claim 79 further characterized by a DSC thermogram exhibiting a peak endotherm at an onset temperature of about 166° C. and a peak maximum at about 183° C.

200. The polymorph of claim 80 further characterized by a DSC thermogram exhibiting a peak endotherm at an onset temperature of about 166° C. and a peak maximum at about 183° C.

201. The polymorph of claim 81 further characterized by a DSC thermogram exhibiting a peak endotherm at an onset temperature of about 166° C. and a peak maximum at about 183° C.

202. The polymorph of claim 82 further characterized by a DSC thermogram exhibiting a peak endotherm at an onset temperature of about 166° C. and a peak maximum at about 183° C.

203. The polymorph of claim 83 further characterized by a DSC thermogram exhibiting a peak endotherm at an onset temperature of about 166° C. and a peak maximum at about 183° C.

204. The polymorph of claim 84 further characterized by a DSC thermogram exhibiting a peak endotherm at an onset temperature of about 166° C. and a peak maximum at about 183° C.

205. The polymorph of claim 85 further characterized by a DSC thermogram exhibiting a peak endotherm at an onset temperature of about 166° C. and a peak maximum at about 183° C.

206. The polymorph of claim 86 further characterized by a DSC thermogram exhibiting a peak endotherm at an onset temperature of about 166° C. and a peak maximum at about 183° C.

207. The polymorph of claim 87 further characterized by a DSC thermogram exhibiting a peak endotherm at an onset temperature of about 166° C. and a peak maximum at about 183° C.

208. The polymorph of claim 88 further characterized by a DSC thermogram exhibiting a peak endotherm at an onset temperature of about 166° C. and a peak maximum at about 183° C.

209. The polymorph of claim 89 further characterized by a DSC thermogram exhibiting a peak endotherm at an onset temperature of about 166° C. and a peak maximum at about 183° C.

210. The polymorph of claim 90 further characterized by a DSC thermogram exhibiting a peak endotherm at an onset temperature of about 166° C. and a peak maximum at about 183° C.

211. The polymorph of claim 91 further characterized by a DSC thermogram exhibiting a peak endotherm at an onset temperature of about 166° C. and a peak maximum at about 183° C.

212. The polymorph of claim 92 further characterized by a DSC thermogram exhibiting a peak endotherm at an onset temperature of about 166° C. and a peak maximum at about 183° C.

213. The polymorph of claim 93 further characterized by a DSC thermogram exhibiting a peak endotherm at an onset temperature of about 166° C. and a peak maximum at about 183° C.

214. The polymorph of claim 94 further characterized by a DSC thermogram exhibiting a peak endotherm at an onset temperature of about 166° C. and a peak maximum at about 183° C.

215. The polymorph of claim 95 further characterized by a DSC thermogram exhibiting a peak endotherm at an onset temperature of about 166° C. and a peak maximum at about 183° C.

216. The polymorph of claim 96 further characterized by a DSC thermogram exhibiting a peak endotherm at an onset temperature of about 166° C. and a peak maximum at about 183° C.

217. The polymorph of claim 97 further characterized by a DSC thermogram exhibiting a peak endotherm at an onset temperature of about 166° C. and a peak maximum at about 183° C.

218. The polymorph of claim 98 further characterized by a DSC thermogram exhibiting a peak endotherm at an onset temperature of about 166° C. and a peak maximum at about 183° C.

219. The polymorph of claim 99 further characterized by a DSC thermogram exhibiting a peak endotherm at an onset temperature of about 166° C. and a peak maximum at about 183° C.

220. The polymorph of claim 100 further characterized by a DSC thermogram exhibiting a peak endotherm at an onset temperature of about 166° C. and a peak maximum at about 183° C.

221. The polymorph of claim 101 further characterized by a DSC thermogram exhibiting a peak endotherm at an onset temperature of about 166° C. and a peak maximum at about 183° C.

222. The polymorph of claim 102 further characterized by a DSC thermogram exhibiting a peak endotherm at an onset temperature of about 166° C. and a peak maximum at about 183° C.

223. The polymorph of claim 103 further characterized by a DSC thermogram exhibiting a peak endotherm at an onset temperature of about 166° C. and a peak maximum at about 183° C.

224. The polymorph of claim 104 further characterized by a DSC thermogram exhibiting a peak endotherm at an onset temperature of about 166° C. and a peak maximum at about 183° C.

225. The polymorph of claim 105 further characterized by a DSC thermogram exhibiting a peak endotherm at an onset temperature of about 166° C. and a peak maximum at about 183° C.

226. The polymorph of claim 106 further characterized by a DSC thermogram exhibiting a peak endotherm at an onset temperature of about 166+ C. and a peak maximum at about 183° C.

227. The polymorph of claim 107 further characterized by a DSC thermogram exhibiting a peak endotherm at an onset temperature of about 166° C. and a peak maximum at about 183° C.

228. The polymorph of claim 108 further characterized by a DSC thermogram exhibiting a peak endotherm at an onset temperature of about 166° C. and a peak maximum at about 183° C.

229. The polymorph of claim 109 further characterized by a DSC thermogram exhibiting a peak endotherm at an onset temperature of about 166° C. and a peak maximum at about 183° C.

230. The polymorph of claim 110 further characterized by a DSC thermogram exhibiting a peak endotherm at an onset temperature of about 166° C. and a peak maximum at about 183° C.

231. The polymorph of claim 111 further characterized by a DSC thermogram exhibiting a peak endotherm at an onset temperature of about 166° C. and a peak maximum at about 183° C.

232. The polymorph of claim 112 further characterized by a DSC thermogram exhibiting a peak endotherm at an onset temperature of about 166° C. and a peak maximum at about 183° C.

233. The polymorph of claim 113 further characterized by a DSC thermogram exhibiting a peak endotherm at an onset temperature of about 166° C. and a peak maximum at about 183° C.

234. The polymorph of claim 114 further characterized by a DSC thermogram exhibiting a peak endotherm at an onset temperature of about 166° C. and a peak maximum at about 183° C.

235. The polymorph of claim 115 further characterized by a DSC thermogram exhibiting a peak endotherm at an onset temperature of about 166° C. and a peak maximum at about 183° C.

236. The polymorph of claim 116 further characterized by a DSC thermogram exhibiting a peak endotherm at an onset temperature of about 166° C. and a peak maximum at about 183° C.

237. The polymorph of claim 117 further characterized by a DSC thermogram exhibiting a peak endotherm at an onset temperature of about 166° C. and a peak maximum at about 183° C.

238. The polymorph of claim 118 further characterized by a DSC thermogram exhibiting a peak endotherm at an onset temperature of about 166° C. and a peak maximum at about 183° C.

239. The polymorph of claim 119 further characterized by a DSC thermogram exhibiting a peak endotherm at an onset temperature of about 166° C. and a peak maximum at about 183° C.

240. The polymorph of claim 120 further characterized by a DSC thermogram exhibiting a peak endotherm at an onset temperature of about 166° C. and a peak maximum at about 183° C.

241. A process for the preparation of form APO-A comprising:
a. combining a compound of formula (1): 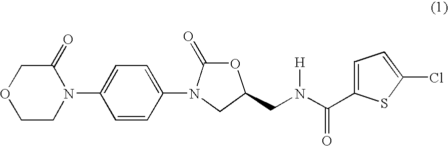
with an organic solvent or a mixture of solvents to form a mixture;
b. heating the mixture;
C. removing undissolved solid to form a solution;
d. promoting crystal growth; and
e. collecting crystals.

242. The process of claim 241 wherein crystals are collected by filtration.

243. The process of claim 241 wherein the starting compound of formula (1) is a polymorphic form other than form APO-A.

244. The process of claim 242 wherein the starting compound of formula (1) is a polymorphic form other than form APO-A.

245. The process of claim 241 wherein the starting compound of formula (1) is polymorphic form modification 1.

246. The process of claim 242 wherein the starting compound of formula (1) is polymorphic form modification 1.

247. The process of claim 241 wherein the starting compound of formula (1) is an amorphous form.

248. The process of claim 242 wherein the starting compound of formula (1) is an amorphous form.

249. The process of claim 241 wherein crystal growth is promoted by cooling the solution.

250. The process of claim 242 wherein crystal growth is promoted by cooling the solution.

251. The process of claim 243 wherein crystal growth is promoted by cooling the solution.

252. The process of claim 244 wherein crystal growth is promoted by cooling the solution.

253. The process of claim 245 wherein crystal growth is promoted by cooling the solution.

254. The process of claim 246 wherein crystal growth is promoted by cooling the solution.

255. The process of claim 247 wherein crystal growth is promoted by cooling the solution.

256. The process of claim 248 wherein crystal growth is promoted by cooling the solution.

257. The process of claim 241 wherein crystal growth is promoted by seeding the solution.

258. The process of claim 242 wherein crystal growth is promoted by seeding the solution.

259. The process of claim 243 wherein crystal growth is promoted by seeding the solution.

260. The process of claim 244 wherein crystal growth is promoted by seeding the solution.

261. The process of claim 245 wherein crystal growth is promoted by seeding the solution.

262. The process of claim 246 wherein crystal growth is promoted by seeding the solution.

263. The process of claim 247 wherein crystal growth is promoted by seeding the solution.

264. The process of claim 248 wherein crystal growth is promoted by seeding the solution.

265. The process of claim 241 wherein crystal growth is promoted by cooling and seeding the solution.

266. The process of claim 242 wherein crystal growth is promoted by cooling and seeding the solution.

267. The process of claim 243 wherein crystal growth is promoted by cooling and seeding the solution.

268. The process of claim 244 wherein crystal growth is promoted by cooling and seeding the solution.

269. The process of claim 245 wherein crystal growth is promoted by cooling and seeding the solution.

270. The process of claim 246 wherein crystal growth is promoted by cooling and seeding the solution.

271. The process of claim 247 wherein crystal growth is promoted by cooling and seeding the solution.

272. The process of claim 248 wherein crystal growth is promoted by cooling and seeding the solution.

273. The process of claim 241 wherein the organic solvent comprises at least one selected from the group consisting of: C3 to C6 ketones, C3 to C4 amides and mixtures thereof.

274. The process of claim 242 wherein the organic solvent comprises at least one selected from the group consisting of: C3 to C6 ketones, C3 to C4 amides and mixtures thereof.

275. The process of claim 243 wherein the organic solvent comprises at least one selected from the group consisting of: C3 to C6 ketones, C3 to C4 amides and mixtures thereof.

276. The process of claim 244 wherein the organic solvent comprises at least one selected from the group consisting of: C3 to C6 ketones, C3 to C4 amides and mixtures thereof.

277. The process of claim 245 wherein the organic solvent comprises at least one selected from the group consisting of: C3 to C6 ketones, C3 to C4 amides and mixtures thereof.

278. The process of claim 246 wherein the organic solvent comprises at least one selected from the group consisting of: C3 to C6 ketones, C3 to C4 amides and mixtures thereof.

279. The process of claim 247 wherein the organic solvent comprises at least one selected from the group consisting of: C3 to C6 ketones, C3 to C4 amides and mixtures thereof.

280. The process of claim 248 wherein the organic solvent comprises at least one selected from the group consisting of: C3 to C6 ketones, C3 to C4 amides and mixtures thereof.

281. The process of claim 249 wherein the organic solvent comprises at least one selected from the group consisting of: C3 to C6 ketones, C3 to C4 amides and mixtures thereof.

282. The process of claim 250 wherein the organic solvent comprises at least one selected from the group consisting of: C3 to C6 ketones, C3 to C4 amides and mixtures thereof.

283. The process of claim 251 wherein the organic solvent comprises at least one selected from the group consisting of: C3 to C6 ketones, C3 to C4 amides and mixtures thereof.

284. The process of claim 252 wherein the organic solvent comprises at least one selected from the group consisting of: C3 to C6 ketones, C3 to C4 amides and mixtures thereof.

285. The process of claim 253 wherein the organic solvent comprises at least one selected from the group consisting of: C3 to C6 ketones, C3 to C4 amides and mixtures thereof.

286. The process of claim 254 wherein the organic solvent comprises at least one selected from the group consisting of: C3 to C6 ketones, C3 to C4 amides and mixtures thereof.

287. The process of claim 255 wherein the organic solvent comprises at least one selected from the group consisting of: C3 to C6 ketones, C3 to C4 amides and mixtures thereof.

288. The process of claim 256 wherein the organic solvent comprises at least one selected from the group consisting of: C3 to C6 ketones, C3 to C4 amides and mixtures thereof.

289. The process of claim 257 wherein the organic solvent comprises at least one selected from the group consisting of: C3 to C6 ketones, C3 to C4 amides and mixtures thereof.

290. The process of claim 258 wherein the organic solvent comprises at least one selected from the group consisting of: C3 to C6 ketones, C3 to C4 amides and mixtures thereof.

291. The process of claim 259 wherein the organic solvent comprises at least one selected from the group consisting of: C3 to C6 ketones, C3 to C4 amides and mixtures thereof.

292. The process of claim 260 wherein the organic solvent comprises at least one selected from the group consisting of: C3 to C6 ketones, C3 to C4 amides and mixtures thereof.

293. The process of claim 261 wherein the organic solvent comprises at least one selected from the group consisting of: C3 to C6 ketones, C3 to C4 amides and mixtures thereof.

294. The process of claim 262 wherein the organic solvent comprises at least one selected from the group consisting of: C3 to C6 ketones, C3 to C4 amides and mixtures thereof.

295. The process of claim 263 wherein the organic solvent comprises at least one selected from the group consisting of: C3 to C6 ketones, C3 to C4 amides and mixtures thereof.

296. The process of claim 264 wherein the organic solvent comprises at least one selected from the group consisting of: C3 to C6 ketones, C3 to C4 amides and mixtures thereof.

297. The process of claim 265 wherein the organic solvent comprises at least one selected from the group consisting of: C3 to C6 ketones, C3 to C4 amides and mixtures thereof.

298. The process of claim 266 wherein the organic solvent comprises at least one selected from the group consisting of: C3 to C6 ketones, C3 to C4 amides and mixtures thereof.

299. The process of claim 267 wherein the organic solvent comprises at least one selected from the group consisting of: C3 to C6 ketones, C3 to C4 amides and mixtures thereof.

300. The process of claim 268 wherein the organic solvent comprises at least one selected from the group consisting of: C3 to C6 ketones, C3 to C4 amides and mixtures thereof.

301. The process of claim 269 wherein the organic solvent comprises at least one selected from the group consisting of: C3 to C6 ketones, C3 to C4 amides and mixtures thereof.

302. The process of claim 270 wherein the organic solvent comprises at least one selected from the group consisting of: C3 to C6 ketones, C3 to C4 amides and mixtures thereof.

303. The process of claim 271 wherein the organic solvent comprises at least one selected from the group consisting of: C3 to C6 ketones, C3 to C4 amides and mixtures thereof.

304. The process of claim 272 wherein the organic solvent comprises at least one selected from the group consisting of: C3 to C6 ketones, C3 to C4 amides and mixtures thereof.

305. The process of claim 241 wherein the organic solvent comprises at least one selected from the group consisting of: 2-butanone, 3-pentanone, methyl-isobutylketone and cyclohexanone.

306. The process of claim 242 wherein the organic solvent comprises at least one selected from the group consisting of: 2-butanone, 3-pentanone, methyl-isobutylketone and cyclohexanone.

307. The process of claim 243 wherein the organic solvent comprises at least one selected from the group consisting of: 2-butanone, 3-pentanone, methyl-isobutylketone and cyclohexanone.

308. The process of claim 244 wherein the organic solvent comprises at least one selected from the group consisting of: 2-butanone, 3-pentanone, methyl-isobutylketone and cyclohexanone.

309. The process of claim 245 wherein the organic solvent comprises at least one selected from the group consisting of: 2-butanone, 3-pentanone, methyl-isobutylketone and cyclohexanone.

310. The process of claim 246 wherein the organic solvent comprises at least one selected from the group consisting of: 2-butanone, 3-pentanone, methyl-isobutylketone and cyclohexanone.

311. The process of claim 247 wherein the organic solvent comprises at least one selected from the group consisting of: 2-butanone, 3-pentanone, methyl-isobutylketone and cyclohexanone.

312. The process of claim 248 wherein the organic solvent comprises at least one selected from the group consisting of: 2-butanone, 3-pentanone, methyl-isobutylketone and cyclohexanone.

313. The process of claim 249 wherein the organic solvent comprises at least one selected from the group consisting of: 2-butanone, 3-pentanone, methyl-isobutylketone and cyclohexanone.

314. The process of claim 250 wherein the organic solvent comprises at least one selected from the group consisting of: 2-butanone, 3-pentanone, methyl-isobutylketone and cyclohexanone.

315. The process of claim 251 wherein the organic solvent comprises at least one selected from the group consisting of: 2-butanone, 3-pentanone, methyl-isobutylketone and cyclohexanone.

316. The process of claim 252 wherein the organic solvent comprises at least one selected from the group consisting of: 2-butanone, 3-pentanone, methyl-isobutylketone and cyclohexanone.

317. The process of claim 253 wherein the organic solvent comprises at least one selected from the group consisting of: 2-butanone, 3-pentanone, methyl-isobutylketone and cyclohexanone.

318. The process of claim 254 wherein the organic solvent comprises at least one selected from the group consisting of: 2-butanone, 3-pentanone, methyl-isobutylketone and cyclohexanone.

319. The process of claim 255 wherein the organic solvent comprises at least one selected from the group consisting of: 2-butanone, 3-pentanone, methyl-isobutylketone and cyclohexanone.

320. The process of claim 256 wherein the organic solvent comprises at least one selected from the group consisting of: 2-butanone, 3-pentanone, methyl-isobutylketone and cyclohexanone.

321. The process of claim 257 wherein the organic solvent comprises at least one selected from the group consisting of: 2-butanone, 3-pentanone, methyl-isobutylketone and cyclohexanone.

322. The process of claim 258 wherein the organic solvent comprises at least one selected from the group consisting of: 2-butanone, 3-pentanone, methyl-isobutylketone and cyclohexanone.

323. The process of claim 259 wherein the organic solvent comprises at least one selected from the group consisting of: 2-butanone, 3-pentanone, methyl-isobutylketone and cyclohexanone.

324. The process of claim 260 wherein the organic solvent comprises at least one selected from the group consisting of: 2-butanone, 3-pentanone, methyl-isobutylketone and cyclohexanone.

325. The process of claim 261 wherein the organic solvent comprises at least one selected from the group consisting of: 2-butanone, 3-pentanone, methyl-isobutylketone and cyclohexanone.

326. The process of claim 262 wherein the organic solvent comprises at least one selected from the group consisting of: 2-butanone, 3-pentanone, methyl-isobutylketone and cyclohexanone.

327. The process of claim 263 wherein the organic solvent comprises at least one selected from the group consisting of: 2-butanone, 3-pentanone, methyl-isobutylketone and cyclohexanone.

328. The process of claim 264 wherein the organic solvent comprises at least one selected from the group consisting of: 2-butanone, 3-pentanone, methyl-isobutylketone and cyclohexanone.

329. The process of claim 265 wherein the organic solvent comprises at least one selected from the group consisting of: 2-butanone, 3-pentanone, methyl-isobutylketone and cyclohexanone.

330. The process of claim 266 wherein the organic solvent comprises at least one selected from the group consisting of: 2-butanone, 3-pentanone, methyl-isobutylketone and cyclohexanone.

331. The process of claim 267 wherein the organic solvent comprises at least one selected from the group consisting of: 2-butanone, 3-pentanone, methyl-isobutylketone and cyclohexanone.

332. The process of claim 268 wherein the organic solvent comprises at least one selected from the group consisting of: 2-butanone, 3-pentanone, methyl-isobutylketone and cyclohexanone.

333. The process of claim 269 wherein the organic solvent comprises at least one selected from the group consisting of: 2-butanone, 3-pentanone, methyl-isobutylketone and cyclohexanone.

334. The process of claim 270 wherein the organic solvent comprises at least one selected from the group consisting of: 2-butanone, 3-pentanone, methyl-isobutylketone and cyclohexanone.

335. The process of claim 271 wherein the organic solvent comprises at least one selected from the group consisting of: 2-butanone, 3-pentanone, methyl-isobutylketone and cyclohexanone.

336. The process of claim 272 wherein the organic solvent comprises at least one selected from the group consisting of: 2-butanone, 3-pentanone, methyl-isobutylketone and cyclohexanone.

337. The process of claim 241 wherein the organic solvent is selected from at least one of the group consisting of: dimethylformamide and dimethylacetamide.

338. The process of claim 242 wherein the organic solvent is selected from at least one of the group consisting of: dimethylformamide and dimethylacetamide.

339. The process of claim 243 wherein the organic solvent is selected from at least one of the group consisting of: dimethylformamide and dimethylacetamide.

340. The process of claim 244 wherein the organic solvent is selected from at least one of the group consisting of: dimethylformamide and dimethylacetamide.

341. The process of claim 245 wherein the organic solvent is selected from at least one of the group consisting of: dimethylformamide and dimethylacetamide.

342. The process of claim 246 wherein the organic solvent is selected from at least one of the group consisting of: dimethylformamide and dimethylacetamide.

343. The process of claim 247 wherein the organic solvent is selected from at least one of the group consisting of: dimethylformamide and dimethylacetamide.

344. The process of claim 248 wherein the organic solvent is selected from at least one of the group consisting of: dimethylformamide and dimethylacetamide.

345. The process of claim 249 wherein the organic solvent is selected from at least one of the group consisting of: dimethylformamide and dimethylacetamide.

346. The process of claim 250 wherein the organic solvent is selected from at least one of the group consisting of: dimethylformamide and dimethylacetamide.

347. The process of claim 251 wherein the organic solvent is selected from at least one of the group consisting of: dimethylformamide and dimethylacetamide.

348. The process of claim 252 wherein the organic solvent is selected from at least one of the group consisting of: dimethylformamide and dimethylacetamide.

349. The process of claim 253 wherein the organic solvent is selected from at least one of the group consisting of: dimethylformamide and dimethylacetamide.

350. The process of claim 254 wherein the organic solvent is selected from at least one of the group consisting of: dimethylformamide and dimethylacetamide.

351. The process of claim 255 wherein the organic solvent is selected from at least one of the group consisting of: dimethylformamide and dimethylacetamide.

352. The process of claim 256 wherein the organic solvent is selected from at least one of the group consisting of: dimethylformamide and dimethylacetamide.

353. The process of claim 257 wherein the organic solvent is selected from at least one of the group consisting of: dimethylformamide and dimethylacetamide.

354. The process of claim 258 wherein the organic solvent is selected from at least one of the group consisting of: dimethylformamide and dimethylacetamide.

355. The process of claim 259 wherein the organic solvent is selected from at least one of the group consisting of: dimethylformamide and dimethylacetamide.

356. The process of claim 260 wherein the organic solvent is selected from at least one of the group consisting of: dimethylformamide and dimethylacetamide.

357. The process of claim 261 wherein the organic solvent is selected from at least one of the group consisting of: dimethylformamide and dimethylacetamide.

358. The process of claim 262 wherein the organic solvent is selected from at least one of the group consisting of: dimethylformamide and dimethylacetamide.

359. The process of claim 263 wherein the organic solvent is selected from at least one of the group consisting of: dimethylformamide and dimethylacetamide.

360. The process of claim 264 wherein the organic solvent is selected from at least one of the group consisting of: dimethylformamide and dimethylacetamide.

361. The process of claim 265 wherein the organic solvent is selected from at least one of the group consisting of: dimethylformamide and dimethylacetamide.

362. The process of claim 266 wherein the organic solvent is selected from at least one of the group consisting of: dimethylformamide and dimethylacetamide.

363. The process of claim 267 wherein the organic solvent is selected from at least one of the group consisting of: dimethylformamide and dimethylacetamide.

364. The process of claim 268 wherein the organic solvent is selected from at least one of the group consisting of: dimethylformamide and dimethylacetamide.

365. The process of claim 269 wherein the organic solvent is selected from at least one of the group consisting of: dimethylformamide and dimethylacetamide.

366. The process of claim 270 wherein the organic solvent is selected from at least one of the group consisting of: dimethylformamide and dimethylacetamide.

367. The process of claim 271 wherein the organic solvent is selected from at least one of the group consisting of: dimethylformamide and dimethylacetamide.

368. The process of claim 272 wherein the organic solvent is selected from at least one of the group consisting of: dimethylformamide and dimethylacetamide.

369. The process of claim 241 wherein the organic solvent comprises methyl-isobutylketone.

370. The process of claim 242 wherein the organic solvent comprises methyl-isobutylketone.

371. The process of claim 243 wherein the organic solvent comprises methyl-isobutylketone.

372. The process of claim 244 wherein the organic solvent comprises methyl-isobutylketone.

373. The process of claim 245 wherein the organic solvent comprises methyl-isobutylketone.

374. The process of claim 246 wherein the organic solvent comprises methyl-isobutylketone.

375. The process of claim 247 wherein the organic solvent comprises methyl-isobutylketone.

376. The process of claim 248 wherein the organic solvent comprises methyl-isobutylketone.

377. The process of claim 249 wherein the organic solvent comprises methyl-isobutylketone.

378. The process of claim 250 wherein the organic solvent comprises methyl-isobutylketone.

379. The process of claim 251 wherein the organic solvent comprises methyl-isobutylketone.

380. The process of claim 252 wherein the organic solvent comprises methyl-isobutylketone.

381. The process of claim 253 wherein the organic solvent comprises methyl-isobutylketone.

382. The process of claim 254 wherein the organic solvent comprises methyl-isobutylketone.

383. The process of claim 255 wherein the organic solvent comprises methyl-isobutylketone.

384. The process of claim 256 wherein the organic solvent comprises methyl-isobutylketone.

385. The process of claim 257 wherein the organic solvent comprises methyl-isobutylketone.

386. The process of claim 258 wherein the organic solvent comprises methyl-isobutylketone.

387. The process of claim 259 wherein the organic solvent comprises methyl-isobutylketone.

388. The process of claim 260 wherein the organic solvent comprises methyl-isobutylketone.

389. The process of claim 261 wherein the organic solvent comprises methyl-isobutylketone.

390. The process of claim 262 wherein the organic solvent comprises methyl-isobutylketone.

391. The process of claim 263 wherein the organic solvent comprises methyl-isobutylketone.

392. The process of claim 264 wherein the organic solvent comprises methyl-isobutylketone.

393. The process of claim 265 wherein the organic solvent comprises methyl-isobutylketone.

394. The process of claim 266 wherein the organic solvent comprises methyl-isobutylketone.

395. The process of claim 267 wherein the organic solvent comprises methyl-isobutylketone.

396. The process of claim 268 wherein the organic solvent comprises methyl-isobutylketone.

397. The process of claim 269 wherein the organic solvent comprises methyl-isobutylketone.

398. The process of claim 270 wherein the organic solvent comprises methyl-isobutylketone.

399. The process of claim 271 wherein the organic solvent comprises methyl-isobutylketone.

400. The process of claim 272 wherein the organic solvent comprises methyl-isobutylketone.

401. The process of claim 241 wherein the organic solvent consists of methyl-isobutylketone.

402. The process of claim 242 wherein the organic solvent consists of methyl-isobutylketone.

403. The process of claim 243 wherein the organic solvent consists of methyl-isobutylketone.

404. The process of claim 244 wherein the organic solvent consists of methyl-isobutylketone.

405. The process of claim 245 wherein the organic solvent consists of methyl-isobutylketone.

406. The process of claim 246 wherein the organic solvent consists of methyl-isobutylketone.

407. The process of claim 247 wherein the organic solvent consists of methyl-isobutylketone.

408. The process of claim 248 wherein the organic solvent consists of methyl-isobutylketone.

409. The process of claim 249 wherein the organic solvent consists of methyl-isobutylketone.

410. The process of claim 250 wherein the organic solvent consists of methyl-isobutylketone.

411. The process of claim 251 wherein the organic solvent consists of methyl-isobutylketone.

412. The process of claim 252 wherein the organic solvent consists of methyl-isobutylketone.

413. The process of claim 253 wherein the organic solvent consists of methyl-isobutylketone.

414. The process of claim 254 wherein the organic solvent consists of methyl-isobutylketone.

415. The process of claim 255 wherein the organic solvent consists of methyl-isobutylketone.

416. The process of claim 256 wherein the organic solvent consists of methyl-isobutylketone.

417. The process of claim 257 wherein the organic solvent consists of methyl-isobutylketone.

418. The process of claim 258 wherein the organic solvent consists of methyl-isobutylketone.

419. The process of claim 259 wherein the organic solvent consists of methyl-isobutylketone.

420. The process of claim 260 wherein the organic solvent consists of methyl-isobutylketone.

421. The process of claim 261 wherein the organic solvent consists of methyl-isobutylketone.

422. The process of claim 262 wherein the organic solvent consists of methyl-isobutylketone.

423. The process of claim 263 wherein the organic solvent consists of methyl-isobutylketone.

424. The process of claim 264 wherein the organic solvent consists of methyl-isobutylketone.

425. The process of claim 265 wherein the organic solvent consists of methyl-isobutylketone.

426. The process of claim 266 wherein the organic solvent consists of methyl-isobutylketone.

427. The process of claim 267 wherein the organic solvent consists of methyl-isobutylketone.

428. The process of claim 268 wherein the organic solvent consists of methyl-isobutylketone.

429. The process of claim 269 wherein the organic solvent consists of methyl-isobutylketone.

430. The process of claim 270 wherein the organic solvent consists of methyl-isobutylketone.

431. The process of claim 271 wherein the organic solvent consists of methyl-isobutylketone.

432. The process of claim 272 wherein the organic solvent consists of methyl-isobutylketone.

433. A product comprising form APO-A made by a process comprising:
a. combining a compound of formula (1): 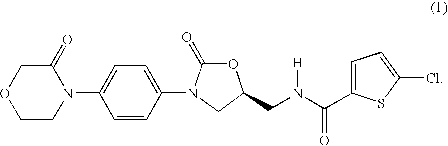
with an organic solvent or a mixture of solvents to form a mixture;
b. heating the mixture;
C. removing undissolved solid to form a solution;
d. promoting crystal growth; and
e. collecting crystals.

434. The product comprising form APO-A of claim 433 wherein the starting compound of formula (1) is polymorphic form modification 1, the organic solvent comprises methyl-isobutylketone, crystal growth is promoted by cooling the solution, and crystals are collected by filtration.

435. The product comprising form APO-A of claim 433 wherein the starting compound of formula (1) is polymorphic form modification I, the organic solvent consists of methyl-isobutylketone, crystal growth is promoted by cooling the solution, and crystals are collected by filtration.

436. A pharmaceutical formulation comprising a polymorphic form of rivaroxaban characterized by an X-ray diffraction pattern comprising a peak at about 39.12 degrees two-theta.

437. A pharmaceutical formulation comprising a polymorphic form of rivaroxaban characterized by an X-ray diffraction pattern comprising a peak at 39.12 degrees two-theta plus or minus 0.2 degree two-theta.

438. A pharmaceutical formulation comprising a polymorphic form of rivaroxaban characterized by an X-ray diffraction pattern comprising a peak at 39.12 degrees two-theta.

439. A pharmaceutical formulation comprising a polymorphic form of rivaroxaban characterized by an X-ray diffraction pattern comprising a peak at about 34.60 degrees two-theta.

440. A pharmaceutical formulation comprising a polymorphic form of rivaroxaban characterized by an X-ray diffraction pattern comprising a peak at 34.60 degrees two-theta plus or minus 0.2 degree two-theta.

441. A pharmaceutical formulation comprising a polymorphic form of rivaroxaban characterized by an X-ray diffraction pattern comprising a peak at 34.60 degrees two-theta.

442. The pharmaceutical formulation of claim 436 wherein the X-ray diffraction pattern further comprises peaks at about 34.60 degrees two-theta, about 25.78 degrees two-theta, about 22.44 degrees two-theta, about 19.27 degrees two-theta and about 8.97 degrees two-theta.

443. The pharmaceutical formulation of claim 437 wherein the X-ray diffraction pattern further comprises peaks at 34.60 degrees two-theta plus or minus 0.2 degree two-theta, 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

444. The pharmaceutical formulation of claim 438 wherein the X-ray diffraction pattern further comprises peaks at 34.60 degrees two-theta, 25.78 degrees two-theta, 22.44 degrees two-theta, 19.27 degrees two-theta and 8.97 degrees two-theta.

445. The pharmaceutical formulation of claim 442 wherein the peak at about 39.12 degrees two-theta has a relative intensity of at least about 8.72%, the peak at about 34.60 degrees two-theta has a relative intensity of at least about 8.45%, the peak at about 25.78 degrees two-theta has a relative intensity of at least about 14.62%, the peak at about 22.44 degrees two-theta has a relative intensity of at least about 10.64%, the peak at about 19.27 degrees two-theta has a relative intensity of at least about 7.42% and the peak at about 8.97 degrees two-theta has a relative intensity of at least about 6.30%.

446. The pharmaceutical formulation of claim 443 wherein the peak at 39.12 degrees two-theta plus or minus 0.2 degree two-theta has a relative intensity of at least about 8.72%, the peak at 34.60 degrees two-theta plus or minus 0.2 degree two-theta has a relative intensity of at least about 8.45%, the peak at 25.78 degrees two-theta plus or minus 0.2 degree two-theta has a relative intensity of at least about 14.62%, the peak at 22.44 degrees two-theta plus or minus 0.2 degree two-theta has a relative intensity of at least about 10.64%, the peak at 19.27 degrees two-theta plus or minus 0.2 degree two-theta has a relative intensity of at least about 7.42% and the peak at 8.97 degrees two-theta plus or minus 0.2 degree two-theta has a relative intensity of at least about 6.30%.

447. The pharmaceutical formulation of claim 444 wherein the peak at 39.12 degrees two-theta has a relative intensity of at least about 8.72%, the peak at 34.60 degrees two-theta has a relative intensity of at least about 8.45%, the peak at 25.78 degrees two-theta has a relative intensity of at least about 14.62%, the peak at 22.44 degrees two-theta has a relative intensity of at least about 10.64%, the peak at 19.27 degrees two-theta has a relative intensity of at least about 7.42% and the peak at 8.97 degrees two-theta has a relative intensity of at least about 6.30%.

448. The pharmaceutical formulation of claim 436 further comprising another polymorphic form of rivaroxaban.

449. The pharmaceutical formulation of claim 437 further comprising another polymorphic form of rivaroxaban.

450. The pharmaceutical formulation of claim 438 further comprising another polymorphic form of rivaroxaban.

451. The pharmaceutical formulation of claim 439 further comprising another polymorphic form of rivaroxaban.

452. The pharmaceutical formulation of claim 440 further comprising another polymorphic form of rivaroxaban.

453. The pharmaceutical formulation of claim 441 further comprising another polymorphic form of rivaroxaban.

454. The pharmaceutical formulation of claim 442 further comprising another polymorphic form of rivaroxaban.

455. The pharmaceutical formulation of claim 443 further comprising another polymorphic form of rivaroxaban.

456. The pharmaceutical formulation of claim 444 further comprising another polymorphic form of rivaroxaban.

457. The pharmaceutical formulation of claim 445 further comprising another polymorphic form of rivaroxaban.

458. The pharmaceutical formulation of claim 446 further comprising another polymorphic form of rivaroxaban.

459. The pharmaceutical formulation of claim 436 further comprising an amorphous form of rivaroxaban.

460. The pharmaceutical formulation of claim 437 further comprising an amorphous form of rivaroxaban.

461. The pharmaceutical formulation of claim 438 further comprising an amorphous form of rivaroxaban.

462. The pharmaceutical formulation of claim 439 further comprising an amorphous form of rivaroxaban.

463. The pharmaceutical formulation of claim 440 further comprising an amorphous form of rivaroxaban.

464. The pharmaceutical formulation of claim 441 further comprising an amorphous form of rivaroxaban.

465. The pharmaceutical formulation of claim 442 further comprising an amorphous form of rivaroxaban.

466. The pharmaceutical formulation of claim 443 further comprising an amorphous form of rivaroxaban.

467. The pharmaceutical formulation of claim 444 further comprising an amorphous form of rivaroxaban.

468. The pharmaceutical formulation of claim 445 further comprising an amorphous form of rivaroxaban.

469. The pharmaceutical formulation of claim 446 further comprising an amorphous form of rivaroxaban.

470. The pharmaceutical formulation of claim 436 further comprising methyl-isobutylketone.

471. The pharmaceutical formulation of claim 437 further comprising methyl-isobutylketone.

472. The pharmaceutical formulation of claim 438 further comprising methyl-isobutylketone.

473. The pharmaceutical formulation of claim 439 further comprising methyl-isobutylketone.

474. The pharmaceutical formulation of claim 440 further comprising methyl-isobutylketone.

475. The pharmaceutical formulation of claim 441 further comprising methyl-isobutylketone.

476. The pharmaceutical formulation of claim 442 further comprising methyl-isobutylketone.

477. The pharmaceutical formulation of claim 443 further comprising methyl-isobutylketone.

478. The pharmaceutical formulation of claim 444 further comprising methyl-isobutylketone.

479. The pharmaceutical formulation of claim 445 further comprising methyl-isobutylketone.

480. The pharmaceutical formulation of claim 446 further comprising methyl-isobutylketone.

481. A composition comprising a polymorphic form of rivaroxaban characterized by an X-ray diffraction pattern comprising a peak at about 39.12 degrees two-theta and another polymorphic form of rivaroxaban.

482. A composition comprising a polymorphic form of rivaroxaban characterized by an X-ray diffraction pattern comprising a peak at 39.12 degrees two-theta plus or minus 0.2 degree two-theta and another polymorphic form of rivaroxaban.

483. A composition comprising a polymorphic form of rivaroxaban characterized by an X-ray diffraction pattern comprising a peak at 39.12 degrees two-theta and another polymorphic form of rivaroxaban.

484. A composition comprising a polymorphic form of rivaroxaban characterized by an X-ray diffraction pattern comprising a peak at about 34.60 degrees two-theta and another polymorphic form of rivaroxaban.

485. A composition comprising a polymorphic form of rivaroxaban characterized by an X-ray diffraction pattern comprising a peak at 34.60 degrees two-theta plus or minus 0.2 degree two-theta and another polymorphic form of rivaroxaban.

486. A composition comprising a polymorphic form of rivaroxaban characterized by an X-ray diffraction pattern comprising a peak at 34.60 degrees two-theta and another polymorphic form of rivaroxaban.

487. The composition of claim 481 wherein the X-ray diffraction pattern further comprises peaks at about 34.60 degrees two-theta, about 25.78 degrees two-theta, about 22.44 degrees two-theta, about 19.27 degrees two-theta and about 8.97 degrees two-theta.

488. The composition of claim 482 wherein the X-ray diffraction pattern further comprises peaks at 34.60 degrees two-theta plus or minus 0.2 degree two-theta, 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

489. The composition of claim 483 wherein the X-ray diffraction pattern further comprises peaks at 34.60 degrees two-theta, 25.78 degrees two-theta, 22.44 degrees two-theta, 19.27 degrees two-theta and 8.97 degrees two-theta.

490. The composition of claim 487 wherein the peak at about 39.12 degrees two-theta has a relative intensity of at least about 8.72%, the peak at about 34.60 degrees two-theta has a relative intensity of at least about 8.45%, the peak at about 25.78 degrees two-theta has a relative intensity of at least about 14.62%, the peak at about 22.44 degrees two-theta has a relative intensity of at least about 10.64%, the peak at about 19.27 degrees two-theta has a relative intensity of at least about 7.42% and the peak at about 8.97 degrees two-theta has a relative intensity of at least about 6.30%.

491. The composition of claim 488 wherein the peak at 39.12 degrees two-theta plus or minus 0.2 degree two-theta has a relative intensity of at least about 8.72%, the peak at 34.60 degrees two-theta plus or minus 0.2 degree two-theta has a relative intensity of at least about 8.45%, the peak at 25.78 degrees two-theta plus or minus 0.2 degree two-theta has a relative intensity of at least about 14.62%, the peak at 22.44 degrees two-theta plus or minus 0.2 degree two-theta has a relative intensity of at least about 10.64%, the peak at 19.27 degrees two-theta plus or minus 0.2 degree two-theta has a relative intensity of at least about 7.42% and the peak at 8.97 degrees two-theta plus or minus 0.2 degree two-theta has a relative intensity of at least about 6.30%.

492. The composition of claim 489 wherein the peak at 39.12 degrees two-theta has a relative intensity of at least about 8.72%, the peak at 34.60 degrees two-theta has a relative intensity of at least about 8.45%, the peak at 25.78 degrees two-theta has a relative intensity of at least about 14.62%, the peak at 22.44 degrees two-theta has a relative intensity of at least about 10.64%, the peak at 19.27 degrees two-theta has a relative intensity of at least about 7.42% and the peak at 8.97 degrees two-theta has a relative intensity of at least about 6.30%.

493. A composition comprising a polymorphic form of rivaroxaban characterized by an X-ray diffraction pattern comprising a peak at about 39.12 degrees two-theta and an amorphous form of rivaroxaban.

494. A composition comprising a polymorphic form of rivaroxaban characterized by an X-ray diffraction pattern comprising a peak at 39.12 degrees two-theta plus or minus 0.2 degree two-theta and an amorphous form of rivaroxaban.

495. A composition comprising a polymorphic form of rivaroxaban characterized by an X-ray diffraction pattern comprising a peak at 39.12 degrees two-theta and an amorphous form of rivaroxaban.

496. A composition comprising a polymorphic form of rivaroxaban characterized by an X-ray diffraction pattern comprising a peak at about 34.60 degrees two-theta and an amorphous form of rivaroxaban.

497. A composition comprising a polymorphic form of rivaroxaban characterized by an X-ray diffraction pattern comprising a peak at 34.60 degrees two-theta plus or minus 0.2 degree two-theta and an amorphous form of rivaroxaban.

498. A composition comprising a polymorphic form of rivaroxaban characterized by an X-ray diffraction pattern comprising a peak at 34.60 degrees two-theta and an amorphous form of rivaroxaban.

499. The composition of claim 493 wherein the X-ray diffraction pattern further comprises peaks at about 34.60 degrees two-theta, about 25.78 degrees two-theta, about 22.44 degrees two-theta, about 19.27 degrees two-theta and about 8.97 degrees two-theta.

500. The composition of claim 494 wherein the X-ray diffraction pattern further comprises peaks at 34.60 degrees two-theta plus or minus 0.2 degree two-theta, 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

501. The composition of claim 495 wherein the X-ray diffraction pattern further comprises peaks at 34.60 degrees two-theta, 25.78 degrees two-theta, 22.44 degrees two-theta, 19.27 degrees two-theta and 8.97 degrees two-theta.

502. The composition of claim 499 wherein the peak at about 39.12 degrees two-theta has a relative intensity of at least about 8.72%, the peak at about 34.60 degrees two-theta has a relative intensity of at least about 8.45%, the peak at about 25.78 degrees two-theta has a relative intensity of at least about 14.62%, the peak at about 22.44 degrees two-theta has a relative intensity of at least about 10.64%, the peak at about 19.27 degrees two-theta has a relative intensity of at least about 7.42% and the peak at about 8.97 degrees two-theta has a relative intensity of at least about 6.30%.

503. The composition of claim 500 wherein the peak at 39.12 degrees two-theta plus or minus 0.2 degree two-theta has a relative intensity of at least about 8.72%, the peak at 34.60 degrees two-theta plus or minus 0.2 degree two-theta has a relative intensity of at least about 8.45%, the peak at 25.78 degrees two-theta plus or minus 0.2 degree two-theta has a relative intensity of at least about 14.62%, the peak at 22.44 degrees two-theta plus or minus 0.2 degree two-theta has a relative intensity of at least about 10.64%, the peak at 19.27 degrees two-theta plus or minus 0.2 degree two-theta has a relative intensity of at least about 7.42% and the peak at 8.97 degrees two-theta plus or minus 0.2 degree two-theta has a relative intensity of at least about 6.30%.

504. The composition of claim 501 wherein the peak at 39.12 degrees two-theta has a relative intensity of at least about 8.72%, the peak at 34.60 degrees two-theta has a relative intensity of at least about 8.45%, the peak at 25.78 degrees two-theta has a relative intensity of at least about 14.62%, the peak at 22.44 degrees two-theta has a relative intensity of at least about 10.64%, the peak at 19.27 degrees two-theta has a relative intensity of at least about 7.42% and the peak at 8.97 degrees two-theta has a relative intensity of at least about 6.30%.

505. A composition comprising a polymorphic form of rivaroxaban characterized by an X-ray diffraction pattern comprising a peak at about 39.12 degrees two-theta and methyl-isobutylketone.

506. A composition comprising a polymorphic form of rivaroxaban characterized by an X-ray diffraction pattern comprising a peak at 39.12 degrees two-theta plus or minus 0.2 degree two-theta and methyl-isobutylketone.

507. A composition comprising a polymorphic form of rivaroxaban characterized by an X-ray diffraction pattern comprising a peak at 39.12 degrees two-theta and methyl-isobutylketone.

508. A composition comprising a polymorphic form of rivaroxaban characterized by an X-ray diffraction pattern comprising a peak at about 34.60 degrees two-theta and methyl-isobutylketone.

509. A composition comprising a polymorphic form of rivaroxaban characterized by an X-ray diffraction pattern comprising a peak at 34.60 degrees two-theta plus or minus 0.2 degree two-theta and methyl-isobutylketone.

510. A composition comprising a polymorphic form of rivaroxaban characterized by an X-ray diffraction pattern comprising a peak at 34.60 degrees two-theta and methyl-isobutylketone.

511. The composition of claim 505 wherein the X-ray diffraction pattern further comprises peaks at about 34.60 degrees two-theta, about 25.78 degrees two-theta, about 22.44 degrees two-theta, about 19.27 degrees two-theta and about 8.97 degrees two-theta.

512. The composition of claim 506 wherein the X-ray diffraction pattern further comprises peaks at 34.60 degrees two-theta plus or minus 0.2 degree two-theta, 25.78 degrees two-theta plus or minus 0.2 degree two-theta, 22.44 degrees two-theta plus or minus 0.2 degree two-theta, 19.27 degrees two-theta plus or minus 0.2 degree two-theta and 8.97 degrees two-theta plus or minus 0.2 degree two-theta.

513. The composition of claim 507 wherein the X-ray diffraction pattern further comprises peaks at 34.60 degrees two-theta, 25.78 degrees two-theta, 22.44 degrees two-theta, 19.27 degrees two-theta and 8.97 degrees two-theta.

514. The composition of claim 511 wherein the peak at about 39.12 degrees two-theta has a relative intensity of at least about 8.72%, the peak at about 34.60 degrees two-theta has a relative intensity of at least about 8.45%, the peak at about 25.78 degrees two-theta has a relative intensity of at least about 14.62%, the peak at about 22.44 degrees two-theta has a relative intensity of at least about 10.64%, the peak at about 19.27 degrees two-theta has a relative intensity of at least about 7.42% and the peak at about 8.97 degrees two-theta has a relative intensity of at least about 6.30%.

515. The composition of claim 512 wherein the peak at 39.12 degrees two-theta plus or minus 0.2 degree two-theta has a relative intensity of at least about 8.72%, the peak at 34.60 degrees two-theta plus or minus 0.2 degree two-theta has a relative intensity of at least about 8.45%, the peak at 25.78 degrees two-theta plus or minus 0.2 degree two-theta has a relative intensity of at least about 14.62%, the peak at 22.44 degrees two-theta plus or minus 0.2 degree two-theta has a relative intensity of at least about 10.64%, the peak at 19.27 degrees two-theta plus or minus 0.2 degree two-theta has a relative intensity of at least about 7.42% and the peak at 8.97 degrees two-theta plus or minus 0.2 degree two-theta has a relative intensity of at least about 6.30%.

516. The composition of claim 513 wherein the peak at 39.12 degrees two-theta has a relative intensity of at least about 8.72%, the peak at 34.60 degrees two-theta has a relative intensity of at least about 8.45%, the peak at 25.78 degrees two-theta has a relative intensity of at least about 14.62%, the peak at 22.44 degrees two-theta has a relative intensity of at least about 10.64%, the peak at 19.27 degrees two-theta has a relative intensity of at least about 7.42% and the peak at 8.97 degrees two-theta has a relative intensity of at least about 6.30%.

517. A composition comprising a crystalline form of rivaroxaban and an organic solvent selected from the group consisting of: C3 to C6 ketones, C3 to C4 amides and mixtures thereof.

518. The composition of claim 517 wherein the organic solvent is at least one selected from the group consisting of: 2-butanone, 3-pentanone, methyl-isobutylketone and cyclohexanone.

519. The composition of claim 517 wherein the organic solvent is at least one selected from the group consisting of: dimethylformamide and dimethylacetamide.

520. The composition of claim 517 wherein the organic solvent comprises methyl-isobutylketone.

521. The composition of claim 517 wherein the organic solvent is methyl-isobutylketone.
